(19) United States
(12) Patent Application Publication
(10) Pub. No.: US 2026/0345945 A1
(43) Pub. Date: Nov. 5, 2026

(54) PORTABLE SOUND THERAPY BOOTH WITH INTEGRATED BIOMETRIC MONITORING

(71) Applicant: Dysruptek LLC, Jacksonville, FL (US)

(72) Inventor: Hamzah A. Shanbari, Jacksonville, FL (US)

(21) Appl. No.: 19/664,765

(22) Filed: Apr. 30, 2026

Related U.S. Application Data
(60) Provisional application No. 63/797,659, filed on Apr. 30, 2025.

(51) Int. Cl. A61M 21/00; G06F 3/01; G06F 3/16; G16H 20/40

(52) U.S. Cl. CPC A61M 21/0094; G16H 20/40; A61M 2021/0022; A61M 2021/0027; A61M 2021/005; G06F 3/011; G06F 3/165

(57) ABSTRACT

An apparatus may comprise a portable booth comprising a weatherproof exterior shell, a modular frame, and antimicrobial interior surfaces. A sound system, a visual display system, a haptic transducer array, and an ambient lighting system may be disposed within the booth and configured to deliver therapeutic audio content, relaxation imagery, vibrotactile stimulation, and chromatic light output across four sensory channels simultaneously. A contactless biometric sensing array may collect physiological data from a user without physical contact. An automated sanitization system may activate upon booth vacancy, comprising at least one of an ultraviolet light emitter and a sanitizing mist dispenser. A controller may receive the physiological data, determine stress indicators, select therapeutic content, and dynamically adjust delivery in real time. The haptic transducer array may operate in a bilateral alternating stimulation pattern synchronized with the audio content. Sessions may be configured for two to fifteen minutes in high-ambient-noise occupational environments.

### RELATED APPLICATION

Under provisions of 35 U.S.C. § 119(e), the Applicant claims benefit of U.S. Provisional Application No. 63/797,659 filed on Apr. 30, 2025, and having inventors in common, which is incorporated herein by reference in its entirety.

It is intended that each of the referenced applications may be applicable to the concepts and embodiments disclosed herein, even if such concepts and embodiments are disclosed in the referenced application with different limitations and configurations and described using different examples and terminology.

### FIELD OF DISCLOSURE

The present disclosure generally relates to occupational health and wellness systems. More specifically, the disclosure pertains to portable therapeutic environments designed to address mental health challenges in high-stress work environments through the integration of audio-visual stimulation with biometric monitoring capabilities.

### BACKGROUND

In some situations, the mental health of construction workers may be significantly impacted by the demanding and stressful nature of their work environment. For example, construction sites may be characterized by high noise levels, physical dangers, long hours, and often limited privacy, creating conditions that may negatively affect psychological wellbeing. Conventional strategies may provide off-site counseling services or periodic mental health check-ups that require workers to seek help outside of their work environment. This approach may not address the immediate need for mental health support directly at construction sites. For example, construction workers may avoid seeking mental health support due to stigma, inconvenience, or inability to access services during their working hours, leading to untreated stress, anxiety, and depression that may worsen over time.

The construction industry has one of the highest rates of suicide among all occupational groups, indicating a critical need for more accessible mental health interventions. Current approaches often fail to consider the unique aspects of construction culture, where displays of vulnerability may be perceived as weakness. Additionally, the transient nature of construction projects means workers frequently change locations, making consistent access to traditional mental health services challenging.

The physical demands of construction work can exacerbate mental health issues, as chronic pain and fatigue contribute to psychological distress. Many workers experience musculoskeletal disorders that, when combined with mental health challenges, create a cycle of worsening physical and psychological symptoms. The conventional approach does not adequately address this mind-body connection.

Furthermore, the hierarchical structure of construction sites can inhibit open communication about mental health concerns. Workers may fear reporting psychological distress could jeopardize their employment status or advancement opportunities. This reluctance is compounded by the predominately male workforce in construction, where traditional gender norms may discourage seeking help for emotional or psychological issues.

Economic pressures also play a role, as construction workers often face financial insecurity due to seasonal work, weather-dependent schedules, and project-based employment. These financial stressors can significantly impact mental wellbeing, yet conventional mental health services rarely integrate financial counseling with psychological support.

The COVID-19 pandemic has introduced additional stressors, including concerns about workplace exposure, project delays, and economic uncertainty, further highlighting the inadequacy of traditional mental health support models for this population.

Several currently available solutions exist for addressing the mental health needs of construction workers, but they each have significant limitations.

Traditional Employee Assistance Programs (EAPs) typically offer off-site counseling services that require workers to seek help outside of their work environment. While these programs provide professional mental health support, they are often underutilized in the construction industry because they require workers to take time away from work, travel to appointments, and overcome the stigma associated with seeking help. Many construction workers avoid using these services due to concerns about confidentiality, potential impact on job security, and the inconvenience of scheduling appointments during limited non-working hours.

Mobile mental health applications represent another approach, offering meditation and stress reduction techniques that workers can access on their personal devices. However, these solutions lack the physical space and environmental control needed in the noisy, chaotic construction environment. The effectiveness of these apps is limited by the constant distractions and lack of privacy on construction sites, making it difficult for workers to engage meaningfully with the digital content.

On-site counselors who periodically visit construction sites provide another solution, but their intermittent presence cannot address immediate mental health needs. The transient nature of construction projects means that by the time a counselor is scheduled to visit, a worker experiencing acute distress may have already reached a crisis point. Additionally, the visible nature of meeting with an on-site counselor can deter workers concerned about being seen by colleagues.

Workplace wellness programs in construction often focus primarily on physical health and safety while neglecting mental wellbeing. These programs typically lack the specialized components needed to address the unique psychological stressors of construction work, such as job insecurity, physical danger, and the pressure to maintain a stoic demeanor.

Peer support programs have been implemented in some construction companies, training workers to recognize signs of distress in colleagues. While these programs help reduce stigma, they do not provide professional intervention, and peer supporters often lack the training to handle complex mental health issues. The effectiveness of these programs is also limited by the hierarchical structure of construction sites, where workers may be reluctant to discuss personal struggles with colleagues.

The inadequacy of these conventional approaches is further compounded by the construction industry's culture, which has historically valued physical toughness over emotional wellbeing. This cultural context creates additional barriers to seeking help, as workers fear being perceived as weak or unreliable if they acknowledge mental health challenges.

There may be a need for accessible mental health interventions directly at construction sites. The construction industry faces challenges that conventional mental health support approaches may fail to address effectively. Construction workers may experience high noise levels, physical dangers, long hours, and limited privacy, creating conditions that may negatively affect psychological wellbeing. Traditional strategies may rely on off-site counseling services or periodic mental health check-ups that require workers to seek help outside their work environment, which many may avoid due to stigma, inconvenience, or inability to access services during working hours.

The construction industry has one of the highest suicide rates among all occupational groups, highlighting the inadequacy of current mental health support models. The industry's culture, which traditionally values physical toughness over emotional wellbeing, creates additional barriers to seeking help. Workers fear being perceived as weak or unreliable if they acknowledge mental health challenges, particularly in a predominantly male workforce where traditional gender norms may discourage seeking help for psychological issues.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The present disclosure provides a portable, self-contained sound therapy booth that may deliver multi-sensory therapeutic content to occupational workers at the point of need, integrating contactless biometric monitoring, adaptive content delivery, automated sanitization, and privacy-preserving data analytics within a single ruggedized, deployable enclosure. The disclosure addresses the absence of immediate, private, and accessible mental health support in high-stress occupational environments such as construction sites, where existing interventions may fail to provide on-site, stigma-free, and technology-enabled stress reduction at the moment of need.

The booth may combine a weatherproof, noise-attenuating enclosure with an audio delivery system, a synchronized visual display system, a contactless biometric sensing array, an automated sanitization system, and a central controller that may orchestrate all subsystems in real time. Contactless sensing technologies may measure heart rate, heart rate variability, respiration, and facial temperature without requiring the user to remove personal protective equipment, enabling use in occupational settings. The controller may compute a composite stress indicator from the collected physiological data, select therapeutic audio and visual content matched to the detected stress state, and dynamically adjust content delivery throughout the session based on continuous biometric feedback. Upon session completion, the sanitization system may automatically treat interior surfaces using UV-C irradiation and antimicrobial mist, confirmed by redundant occupancy detection, before the booth is made available to the next user. Anonymized session data may be processed by a machine learning engine, aggregated into workforce wellness metrics, and transmitted via encrypted wireless communication to external occupational health monitoring systems, enabling data-driven occupational health program assessment without exposing individual user information. The modular, portable structure may support disassembly, transport, and redeployment as project locations change. The result may be on-site stress reduction delivered privately, hygienically, and without requiring workers to leave the job site.

The multi-sensory therapy delivery subsystem may include a haptic transducer array configured to deliver vibrotactile stimulation and an ambient lighting system configured to deliver chromatic light output, both synchronized with the therapeutic audio and visual content to provide coordinated multi-modal stimulation. The haptic transducer array may operate in a bilateral alternating stimulation pattern to enhance therapeutic effect. Sessions may be configured for short durations of between two and fifteen minutes, enabling effective stress reduction within high-ambient-noise occupational environments without requiring workers to leave the worksite for extended periods.

In a first aspect, an apparatus may include a portable booth having a weatherproof exterior shell, a modular frame, and antimicrobial interior surfaces. A sound system disposed within the portable booth may be configured to deliver therapeutic audio content, and a visual display system disposed within the booth may be configured to display relaxation imagery synchronized with the therapeutic audio content. A haptic transducer array disposed within the portable booth may be configured to deliver vibrotactile stimulation to a user, and an ambient lighting system disposed within the portable booth may be configured to deliver chromatic and intensity-variable light output synchronized with the therapeutic audio content and the relaxation imagery. A contactless biometric sensing array positioned within the portable booth may be configured to collect physiological data from a user without physical contact with the user. An automated sanitization system including at least one of an ultraviolet light emitter and a sanitizing mist dispenser may be activated upon detection of booth vacancy following a user session. A controller may be communicatively coupled to the sound system, the visual display system, the haptic transducer array, the ambient lighting system, the contactless biometric sensing array, and the automated sanitization system. The controller may receive the physiological data from the contactless biometric sensing array to determine one or more stress indicators from the physiological data, and select therapeutic content based on the determined stress indicators. The controller may further deliver the selected therapeutic content through the sound system, the visual display system, the haptic transducer array and/or the ambient lighting system in a coordinated, synchronized manner, and dynamically adjust the therapeutic content in real time based on changes in the physiological data monitored during the user session.

In another aspect, a method may include detecting presence of a user within a portable booth deployed at a worksite. Physiological data may be collected from the detected user, without physical contact with the user, using a plurality of contactless biometric sensors positioned within the portable booth. One or more stress indicators may be determined from the collected physiological data, and therapeutic audio content and synchronized visual content may be selected based on the determined stress indicators. The method may further include delivering the therapeutic audio content through a sound system disposed within the portable booth, displaying the synchronized visual content through a visual display system disposed within the portable booth, activating a haptic transducer array to deliver vibrotactile stimulation synchronized with the therapeutic audio content, and activating an ambient lighting system to deliver chromatic light output synchronized with the therapeutic audio content and the synchronized visual content. Changes in the physiological data may be monitored during a therapy session, and adjustments may be made to the therapeutic audio content, the synchronized visual content, the vibrotactile stimulation, and/or the chromatic light output in real time based on the monitored changes. The method may conclude by activating an automated sanitization system within the portable booth upon detecting vacancy of the portable booth following completion of the therapy session.

In yet another aspect, a system may include a portable booth comprising a weatherproof exterior shell, a modular frame configured for transport and reassembly between worksites, and antimicrobial interior surfaces. A multi-sensory therapy delivery subsystem disposed within the booth may include a sound system, a visual display system, a haptic transducer array, and an ambient lighting system configured to deliver coordinated, synchronized therapeutic audio content, visual content, vibrotactile stimulation, and chromatic light output. A contactless biometric monitoring subsystem having a plurality of non-contact sensors may be configured to collect physiological data from a user within the portable booth without physical contact with the user. An automated sanitization subsystem may disinfect interior surfaces of the portable booth between user sessions. The system may include a data processing subsystem configured to determine stress indicators from the physiological data, select and deliver therapeutic content through the multi-sensory therapy delivery subsystem based on the determined stress indicators, and dynamically adjust the therapeutic content based on changes in the physiological data monitored during a therapy session. The data processing subsystem may further store anonymized session data, and generate workforce wellness analytics from the anonymized session data. A secure communication module may be configured to transmit encrypted wellness metrics to an external occupational health system.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1A is a block diagram illustrating an overall system architecture of a portable sound therapy booth and its interconnected subsystems and application modules.

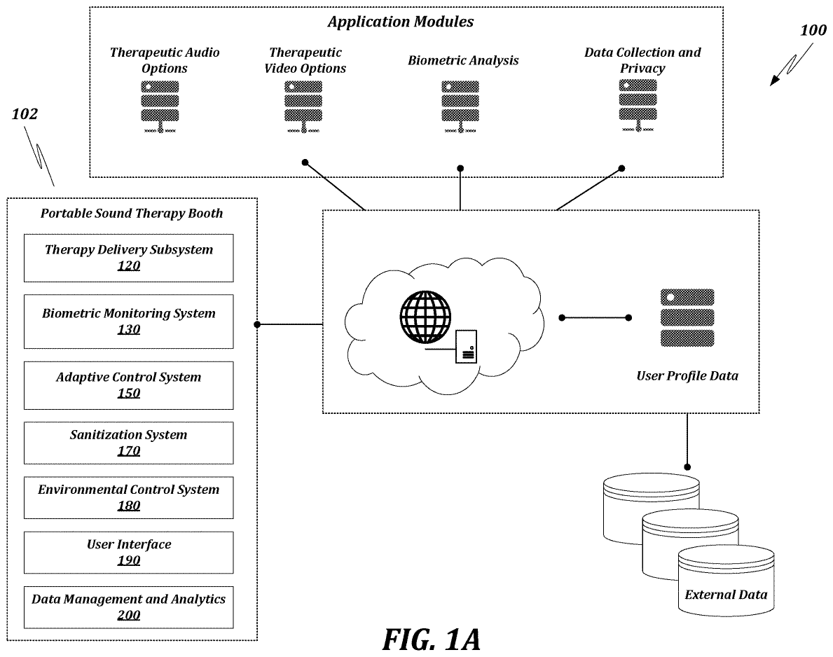

FIG. 1B is a block diagram illustrating a therapy delivery subsystem of the portable sound therapy booth.

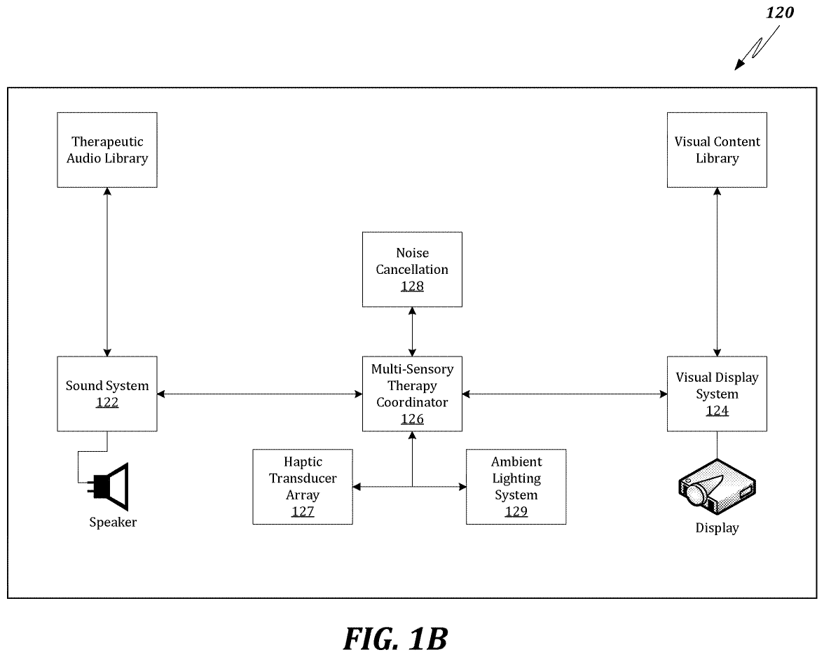

FIG. 1C is a block diagram illustrating a biometric monitoring system of the portable sound therapy booth.

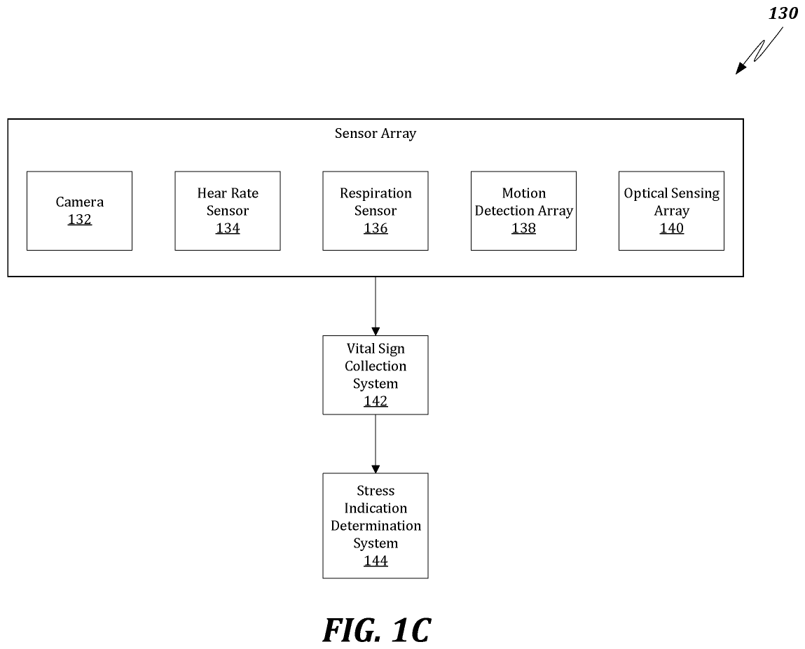

FIG. 1D is a block diagram illustrating an adaptive control system of the portable sound therapy booth.

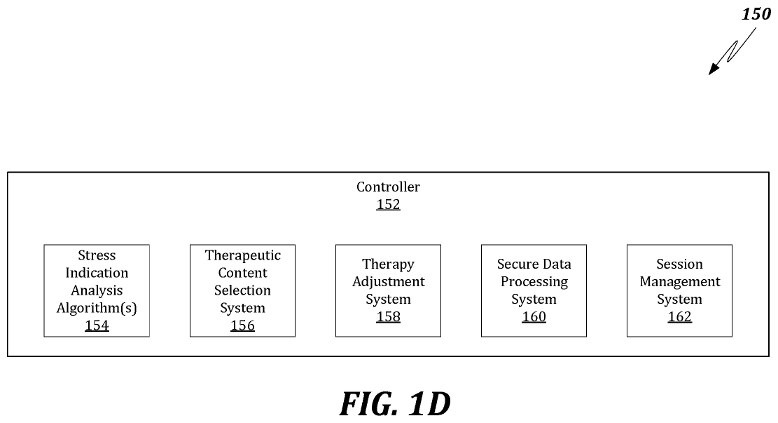

FIG. 1E is a block diagram illustrating a sanitization system of the portable sound therapy booth.

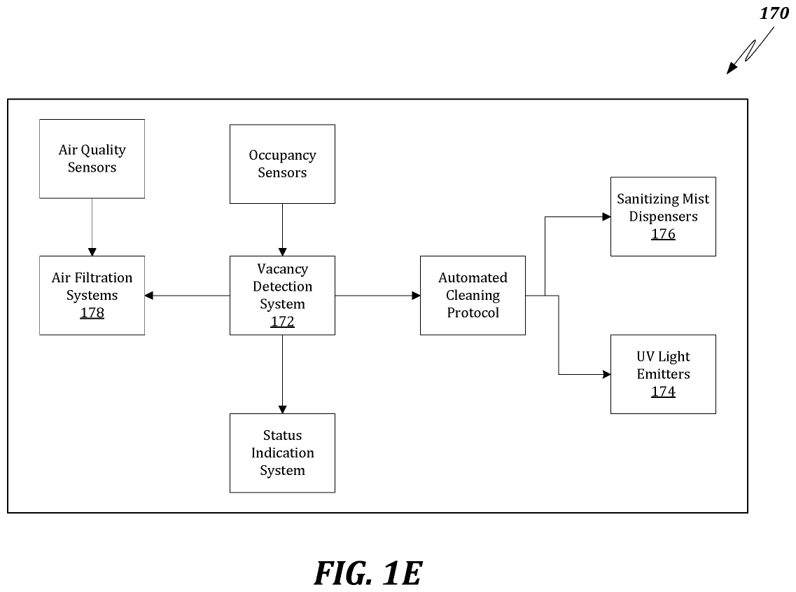

FIG. 1F is a block diagram illustrating an environmental control system of the portable sound therapy booth.

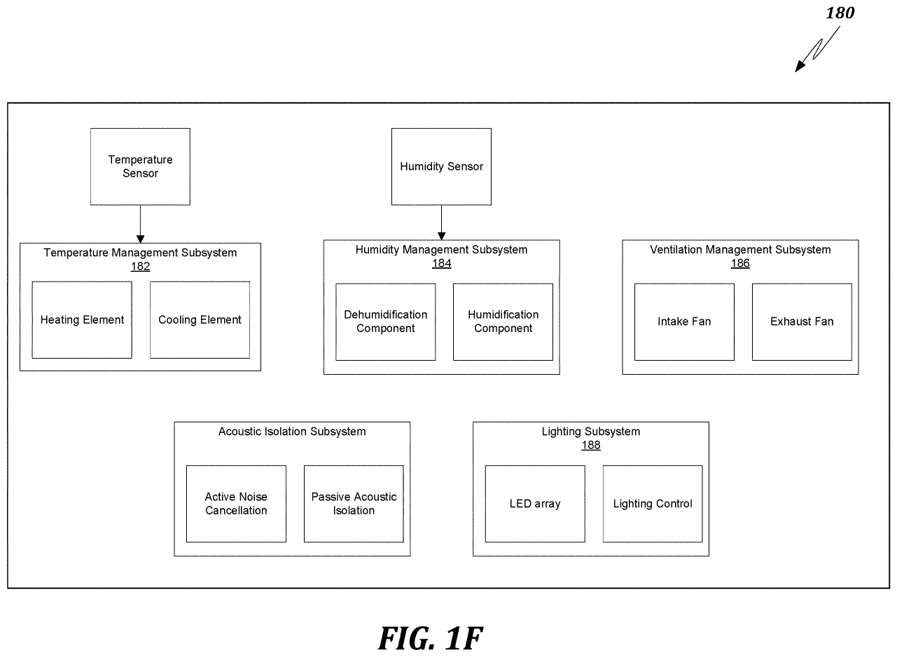

FIG. 1G is a block diagram illustrating a user interface of the portable sound therapy booth.

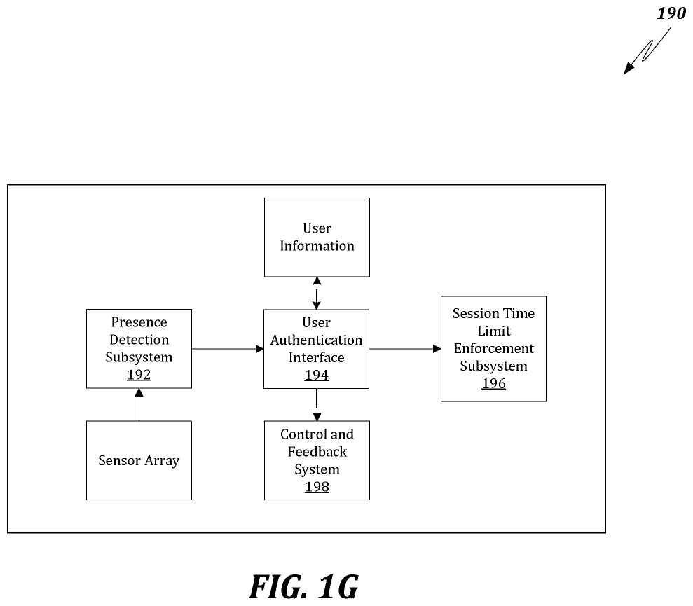

FIG. 1H is a block diagram illustrating a data management and analytics system of the portable sound therapy booth.

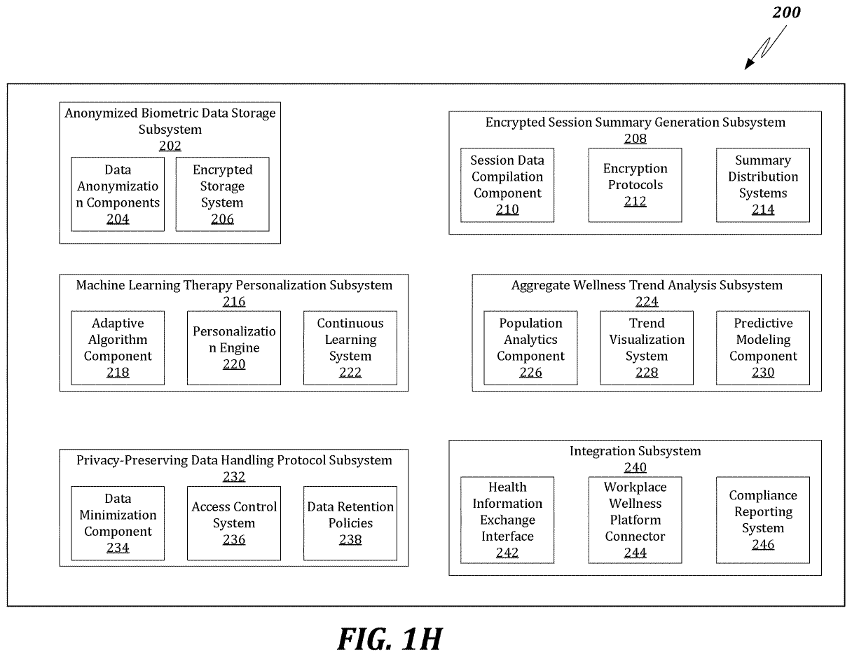

FIG. 2 is a perspective interior view of the portable sound therapy booth showing an arrangement of internal components.

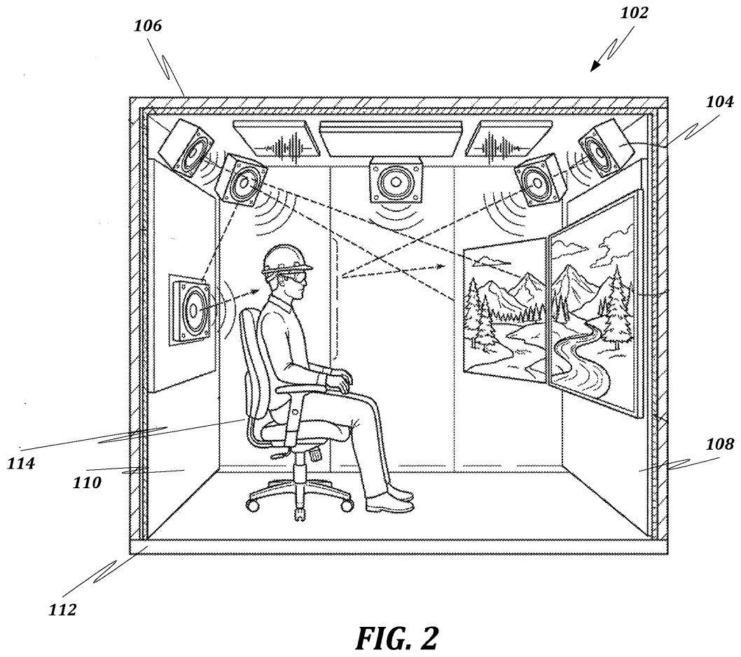

FIG. 3 is a flowchart illustrating a method of operating the portable sound therapy booth.

FIG. 4 is a block diagram illustrating a computing device for implementing the portable sound therapy booth system.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely to provide a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such a term to mean based on the contextual use of the term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subject matter disclosed under the header.

A portable therapy booth system may provide immediate, on-site therapeutic intervention for workers in high-stress environments. The system may comprise a weatherproof modular booth structure with antimicrobial surfaces configured for deployment at construction sites and other challenging work environments. The booth may provide a controlled therapeutic environment that addresses the unique mental health challenges faced by construction workers and similar occupational groups.

The system may include integrated sound and visual therapy delivery systems disposed within the portable booth. The sound system may be configured to deliver therapeutic audio content, while a visual display system may be configured to display relaxation imagery synchronized with the therapeutic audio content. The multi-sensory approach may provide immersive therapeutic experiences that can effectively reduce stress and anxiety in noisy, chaotic work environments.

A contactless biometric sensing array may be positioned within the portable booth to monitor physiological data from users without requiring physical contact. The sensing array may collect real-time data on stress indicators such as heart rate, respiration patterns, and thermal signatures while accommodating personal protective equipment commonly worn by construction workers.

A controller may be communicatively coupled to the therapy delivery systems and biometric sensing array to provide adaptive therapy content selection based on physiological data. The controller may receive physiological data, determine stress indicators from the data, select appropriate therapeutic content based on the determined stress indicators, and dynamically adjust the therapeutic content in real time based on changes in physiological data monitored during user sessions.

An automated sanitization system may be configured for shared-use hygiene between user sessions. The sanitization system may activate upon detection of booth vacancy following a user session and may comprise ultraviolet light emitters and sanitizing mist dispensers to disinfect interior surfaces, ensuring safe shared use in workplace environments.

The present disclosure includes many aspects and features. Moreover, while many aspects and features relate to, and are described in, the context of a portable therapy booth system, embodiments of the present disclosure are not limited to use only in this context.

### I. Platform Overview

This overview is provided to introduce a selection of concepts in a simplified form that are further described below. This overview is not intended to identify key features or essential features of the claimed subject matter. Nor is this overview intended to be used to limit the claimed subject matter's scope.

The portable sound therapy booth is a self-contained, deployable wellness unit that brings immediate, private mental health support directly to workers in high-stress occupational environments, such as construction sites. Rather than requiring a worker to leave the job site, schedule an appointment, or openly seek help from a colleague or supervisor, the booth functions like a private wellness station that a worker can step into during a break. The experience inside the booth is designed to stand in sharp contrast to the loud, chaotic, and physically demanding environment outside.

The booth is a compact, weatherproof enclosure built from a modular frame with antimicrobial interior surfaces. Because construction sites are temporary and move as projects progress, the booth is designed to be disassembled, transported, and reassembled at new locations without specialized tools or dedicated installation crews. The exterior is built to withstand dust, debris, rain, and temperature extremes common to outdoor work environments.

Inside the booth, a sound system, a visual display system, a haptic transducer array, and an ambient lighting system work together to deliver a coordinated, synchronized multi-sensory therapeutic experience across up to four simultaneous output channels. The sound system delivers calming audio content, which may include nature sounds, guided breathing exercises, instrumental music, or specialized audio tones designed to guide the brain toward a more relaxed state. The visual display shows relaxation imagery that is synchronized with the audio, so that what the user sees and hears work together rather than competing for attention. The haptic transducer array delivers vibrotactile stimulation coordinated with the audio and visual content, and may operate in a bilateral alternating stimulation pattern in which a left-side transducer and a right-side transducer are activated in alternating sequence synchronized with the therapeutic audio content. The ambient lighting system delivers chromatic and intensity-variable light output synchronized with all other channels. All four channels are coordinated and controlled together by the controller, which adjusts each channel in real time based on the user's physiological state, creating an immersive multi-modal environment that is meaningfully different from the sensory overload of the surrounding worksite. The combination creates an immersive environment that is meaningfully different from the sensory overload of the surrounding worksite.

A key feature of the booth is its ability to monitor the user's physiological state without requiring the user to touch any device, attach any sensor, or remove any personal protective equipment. A set of contactless sensors positioned within the booth continuously measures indicators of stress, including heart rate, breathing rate, and skin temperature patterns, simply by observing the user while they sit in the booth. This non-contact approach is important in a construction environment where workers routinely wear gloves, hard hats, and other protective gear.

A controller inside the booth receives the data from these sensors in real time and uses it to make decisions about the therapeutic content being delivered across all four sensory output channels. If the sensors indicate that a user is highly stressed at the start of a session, the controller selects content appropriate for that stress level and configures the audio, visual, haptic, and lighting outputs accordingly. As the session progresses and the user's physiological readings change, the controller continuously adjusts all four channels in a coordinated manner to match the user's evolving state. This adaptive multi-channel approach means that each session is personalized to the individual user's actual physiological condition at that moment, rather than delivering a fixed, one-size-fits-all program through any single sensory channel.

When a user exits the booth, the system detects the vacancy and automatically initiates a sanitization cycle. Ultraviolet light emitters and sanitizing mist dispensers work together to disinfect the interior surfaces, so that the next user enters a clean environment. This automated hygiene process is particularly important in a shared-use setting where many workers from different trades may use the booth throughout a workday.

Beyond the individual session, the booth collects anonymized data over time. Personal identifying information is removed from the data before it is stored or transmitted. The system analyzes this anonymized data to identify patterns in workforce stress levels, evaluate which therapeutic content is most effective, and generate reports for health and safety managers. These reports can help employers understand when and where stress is highest across their workforce, enabling more informed decisions about scheduling, resource allocation, and wellness program design, all without exposing any individual worker's personal health information.

The booth also includes environmental controls that regulate temperature, humidity, and ventilation inside the enclosure, as well as active noise cancellation hardware that reduces the intrusion of external construction site noise into the therapeutic space. An emergency alert system monitors for abnormal biometric readings and can generate an alert if a potential health emergency is detected. Emergency exit mechanisms ensure that a user can always leave the booth quickly, including in the event of a complete power failure.

In summary, the portable sound therapy booth is a practical, deployable tool that delivers personalized, evidence-informed stress relief directly at the worksite, monitors its own effectiveness in real time through contactless physiological sensing, maintains hygiene automatically between users, and generates actionable workforce wellness data, all within a rugged, portable enclosure designed for the realities of demanding occupational environments.

Embodiments of the present disclosure may comprise methods, systems, and a computer readable medium comprising, but not limited to, at least one of the following:
A. A Booth
B. A Therapy Delivery Subsystem
C. A Biometric Monitoring System
D. An Adaptive Control System
E. An Automated Sanitization System

In some embodiments, the present disclosure may provide an additional set of modules for further facilitating the software and hardware platform. The additional set of modules may comprise, but not be limited to:
F. An Environmental Control System
G. A User Interface and Session Management System
H. A Data Management and Analytics Subsystem

Details with regards to each module are provided below. Although modules are disclosed with specific functionality, it should be understood that functionality may be shared between modules, with some functions split between modules, while other functions duplicated by the modules. Furthermore, the name of each module should not be construed as limiting upon the functionality of the module. Moreover, each component disclosed within each module can be considered independently, without the context of the other components within the same module or different modules. Each component may contain functionality defined in other portions of this specification. Each component disclosed for one module may be mixed with the functionality of other modules. In the present disclosure, each component can be claimed on its own and/or interchangeably with other components of other modules.

The following depicts an example of a method of a plurality of methods that may be performed by at least one of the aforementioned modules, or components thereof. Various hardware components may be used at the various stages of the operations disclosed with reference to each module. For example, although methods may be described to be performed by a single computing device, it should be understood that, in some embodiments, different operations may be performed by different networked elements in operative communication with the computing device. For example, at least one computing device 400 may be employed in the performance of some or all of the stages disclosed with regard to the methods. Similarly, an apparatus may be employed in the performance of some or all of the stages of the methods. As such, the apparatus may comprise at least those architectural components as found in computing device 400.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in orders that differ from the ones disclosed below. Moreover, various stages may be added or removed without altering or departing from the fundamental scope of the depicted methods and systems disclosed herein.

Consistent with embodiments of the present disclosure, a method may be performed by at least one of the modules disclosed herein. The method may be embodied as, for example, but not limited to, computer instructions which, when executed, perform the method. The method may comprise the following stages:
detecting presence of a user within a portable booth deployed at a worksite;
collecting physiological data from the user using a plurality of contactless biometric sensors positioned within the portable booth, wherein the physiological data is collected without physical contact with the user;
determining one or more stress indicators from the collected physiological data;
selecting therapeutic audio content and synchronized visual content based on the determined stress indicators;
delivering the therapeutic audio content through a sound system disposed within the portable booth and displaying the synchronized visual content through a visual display system disposed within the portable booth, activating a haptic transducer array to deliver vibrotactile stimulation synchronized with the therapeutic audio content, and activating an ambient lighting system to deliver chromatic light output synchronized with the therapeutic audio content and the synchronized visual content;
monitoring changes in the physiological data during a therapy session;
adjusting one or more of the therapeutic audio content, the synchronized visual content, the vibrotactile stimulation, and the chromatic light output in real time based on the monitored changes; and
activating an automated sanitization system within the portable booth upon detecting vacancy of the portable booth following completion of the therapy session.

Although the aforementioned method has been described to be performed by the platform 100, it should be understood that computing device 400 may be used to perform the various stages of the method. Furthermore, in some embodiments, different operations may be performed by different networked elements in operative communication with computing device 400. For example, a plurality of computing devices may be employed in the performance of some or all of the stages in the aforementioned method. Moreover, a plurality of computing devices may be configured much like a single computing device 400. Similarly, an apparatus may be employed in the performance of some or all stages in the method. The apparatus may also be configured much like computing device 400.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

### II. Platform Configuration

The therapy booth system 100 may comprise an integrated architecture of interconnected subsystems designed to provide autonomous therapeutic intervention in challenging work environments. The system 100 may be configured as a self-contained unit capable of deployment at construction sites, industrial facilities, and other high-stress work locations where immediate mental health support may be beneficial.

The portable booth structure may comprise a modular frame and weatherproof shell designed for rapid deployment and environmental durability. The modular frame may provide structural integrity while enabling efficient transport and assembly at various work sites. The weatherproof shell may protect internal components from dust, moisture, temperature extremes, and other environmental hazards commonly encountered at construction sites. The booth structure may include antimicrobial interior surfaces to maintain hygiene standards for shared use applications.

The multi-modal therapy delivery subsystem may comprise integrated audio, visual, haptic, and lighting components configured to provide synchronized therapeutic content across up to four coordinated sensory output channels. The audio subsystem may include speakers, amplifiers, and noise cancellation hardware positioned to deliver therapeutic audio content while isolating users from external environmental noise. The visual subsystem may include display screens, projection systems, and lighting controls configured to present relaxation imagery and ambient lighting synchronized with audio content to create immersive therapeutic experiences. The haptic subsystem may include a haptic transducer array comprising vibrotactile actuators positioned within the seating element and along interior wall surfaces to deliver vibrotactile stimulation coordinated with audio and visual content. The ambient lighting subsystem may include LED light arrays configured to deliver chromatic and intensity-variable light output synchronized with the therapeutic audio content and the relaxation imagery. The controller 152 may be communicatively coupled to all four subsystems and configured to coordinate delivery timing, intensity, and content transitions across the audio, visual, haptic, and ambient lighting channels simultaneously to produce a unified multi-modal therapeutic output.

The contactless biometric monitoring subsystem may comprise sensing arrays positioned throughout the booth interior to collect physiological data without requiring physical contact with users. The sensing array may include thermal imaging cameras configured to detect facial temperature patterns associated with physiological stress responses, optical sensors configured to measure heart rate by detecting skin color variations caused by blood volume changes in facial capillaries using remote photoplethysmography techniques, and motion sensors configured to detect respiration rate through non-contact measurement of chest wall displacement. The contactless design may accommodate personal protective equipment commonly worn by construction workers while maintaining accurate physiological monitoring capabilities. Each sensor type may operate independently and in combination to provide a composite physiological data stream from which stress indicators may be determined.

The automated sanitization subsystem may comprise disinfection hardware configured to activate between user sessions. The subsystem may include ultraviolet light emitters positioned to irradiate interior surfaces, sanitizing mist dispensers for comprehensive surface treatment, and air filtration systems for maintaining clean air quality. Occupancy sensors may trigger sanitization cycles upon detection of booth vacancy to ensure hygienic conditions for subsequent users.

The occupancy detection may implement at least two independent detection modalities to ensure reliable sanitization safety and prevent false triggering. Primary occupancy detection may utilize passive infrared motion sensors positioned at multiple booth locations to detect human body heat signatures and movement patterns. Secondary occupancy verification may employ weight-sensitive floor sensors that detect human presence through pressure distribution analysis and can distinguish between equipment placement and human occupancy.

Tertiary detection systems may include door position sensors and optical beam-break detectors that monitor booth entry and exit events to maintain accurate occupancy status. Detection fusion algorithms may require confirmation from at least two independent modalities before initiating sanitization cycles, with override capabilities available only through manual safety interlocks. The system may implement occupancy timeout protocols that automatically verify booth vacancy after 10 minutes of inactivity before enabling sanitization systems, ensuring user safety even during extended rest periods or emergency situations.

The control and data processing subsystem may comprise a central controller communicatively coupled to all booth subsystems. The controller may receive physiological data from the biometric monitoring subsystem, analyze stress indicators using embedded algorithms, select appropriate therapeutic content from stored libraries, and coordinate delivery through the therapy subsystems. The controller may provide real-time adaptation of therapeutic content based on ongoing physiological monitoring during user sessions.

Power management and connectivity systems may provide operational support for all booth subsystems. The power management system may include connections to site electrical infrastructure, energy-efficient component design, and backup power capabilities for continuous operation. The connectivity system may provide wireless communication capabilities for remote monitoring, content updates, and integration with external occupational health management systems while maintaining user privacy and data security.

The system may further comprise power and deployment subsystems configured to enable flexible installation and reliable operation across diverse construction site environments while maintaining energy efficiency and operational continuity.

A site power infrastructure compatibility subsystem may be configured to interface with standard construction site electrical systems and accommodate varying power supply conditions. Universal power input components may accept multiple voltage configurations including 120V, 240V, and 480V three-phase power commonly available at construction sites. Power conditioning systems may regulate incoming electrical supply to provide stable power delivery despite voltage fluctuations and electrical noise typical of construction environments. Load management circuits may automatically adjust system power consumption based on available site power capacity to prevent electrical system overloading.

Energy-efficient component design subsystems may be configured to minimize power consumption while maintaining full therapeutic functionality. Low-power LED lighting systems may provide therapeutic ambiance lighting while consuming minimal electrical energy compared to traditional lighting technologies. Efficient HVAC components 262 may utilize variable-speed fans and smart temperature control to optimize climate management energy usage. Power management controllers may implement sleep modes and automatic shutdown protocols to reduce standby power consumption during periods of booth inactivity.

Backup power option subsystems may be configured to ensure continuous therapeutic system operation during power outages or electrical system failures. Battery backup systems may provide uninterrupted power for essential systems including biometric monitoring, emergency communications, and user safety systems during temporary power interruptions. Generator interface connections may enable rapid connection to portable generators for extended operation during prolonged power outages. Power transfer switches may automatically transition between primary site power and backup power sources without interrupting ongoing therapeutic sessions.

Transportable design subsystems may be configured to enable efficient relocation of the therapeutic booth between multiple construction sites. Modular frame construction may utilize lightweight aluminum or steel components that can be disassembled and reassembled without specialized tools or equipment. Standardized connection interfaces may enable rapid system setup and breakdown while maintaining structural integrity and weatherproofing. Protective packaging systems 280 may safeguard sensitive electronic components during transport and storage between deployment locations.

Trailer-mounting capability subsystems may be configured to provide mobile therapeutic services that can be rapidly deployed to remote construction sites or temporary work locations. Trailer integration frameworks may secure the booth structure to standard construction trailers while maintaining structural stability during transport and operation. Mobile utility connections may provide standardized interfaces for electrical, water, and communication services at temporary deployment sites. Stabilization and leveling systems may ensure proper booth operation on uneven terrain and provide vibration isolation during mobile deployment applications.

Accordingly, embodiments of the present disclosure provide a software and hardware platform comprised of a distributed set of computing elements, including, but not limited to:

### A. A Booth

The booth 102 may comprise a modular frame 104 designed for portability and efficient site deployment. The modular frame 104 may include lightweight aluminum or steel structural members that can be assembled and disassembled without specialized tools or equipment. The frame 104 may feature interlocking joints and quick-connect fasteners that enable rapid assembly by construction personnel. The modular design may allow the booth 102 to be transported in compact sections and assembled on-site within a predetermined time period, such as thirty minutes or less.

Weatherproof exterior panels 106 may be coupled to the modular frame 104 to provide construction site durability. The exterior panels 106 may comprise composite materials resistant to impact, moisture, temperature extremes, and chemical exposure commonly encountered at construction sites. The panels 106 may include insulation layers to maintain interior temperature control and acoustic dampening properties to reduce external noise transmission. Sealed joints between panels 106 may prevent dust and moisture infiltration while maintaining structural integrity under wind loads and vibration.

Antimicrobial interior surfaces 108 may line the interior walls, ceiling, and floor of the booth 102. The antimicrobial surfaces 108 may comprise materials treated with antimicrobial agents or coatings that inhibit bacterial and viral growth. The surfaces 108 may be configured for easy cleaning and disinfection between user sessions. The antimicrobial properties may remain effective over extended periods of use and exposure to sanitization procedures.

Glass panels 110 may be integrated into the booth structure to provide natural lighting while maintaining environmental protection. The glass panels 110 may include protective elements such as impact-resistant glazing, dust-resistant seals, and debris shields configured to withstand construction site conditions. The panels 110 may be positioned to provide visibility for safety monitoring while preserving user privacy during therapeutic sessions.

Anchoring components 112 may be configured to secure the booth 102 to various site surfaces including concrete, asphalt, gravel, and temporary platforms. The anchoring components 112 may include adjustable feet, ground anchors, and tie-down systems that accommodate uneven terrain and varying surface conditions. The components 112 may provide stability against wind loads and site vibrations while enabling relocation as construction projects progress.

Ergonomic seating 114 may be positioned within the booth 102 to provide comfortable user positioning during therapeutic sessions. The seating 114 may include adjustable height and back support to accommodate users of varying sizes while wearing personal protective equipment. The seating 114 may comprise materials that are easy to clean and resistant to wear from repeated use.

Calming interior design elements may be integrated throughout the booth interior to create a therapeutic environment. The design elements may include soft lighting fixtures, natural color schemes, textured wall treatments, and acoustic panels that reduce harsh reflections. The elements may be selected to provide visual and tactile contrast to typical construction site environments while maintaining durability and ease of maintenance.

### B. A Therapy Delivery Subsystem

The therapy delivery subsystem 120 may comprise integrated audio, visual, haptic, and ambient lighting components configured to provide coordinated, synchronized therapeutic content across up to four sensory output channels within the booth 102. The subsystem 120 may be communicatively coupled to the controller to enable coordinated delivery of multi-sensory therapeutic experiences tailored to individual user needs and stress indicators.

A sound system 122 may be disposed within the booth 102 to deliver therapeutic audio content. The sound system 122 may include speakers positioned at multiple locations within the booth interior to create immersive audio environments. In some embodiments, the sound system 122 may include a stereo speaker configuration comprising a left-channel speaker and a right-channel speaker positioned to deliver independent audio signals to the left and right ears of a seated user, enabling delivery of binaural beat audio content in which a first tone frequency is delivered through the left-channel speaker and a second tone frequency, differing from the first tone frequency by a predetermined beat frequency, is delivered through the right-channel speaker. The beat frequency perceived by the user may correspond to a target brainwave entrainment frequency selected based on the determined stress indicators. In some embodiments, the sound system 122 may additionally or alternatively include a headphone output interface configured to deliver binaural beat audio content through personal headphones, providing enhanced channel separation for binaural beat delivery in environments where speaker-based stereo separation may be insufficient. Audio amplifiers may be configured to drive the speakers with sufficient power to overcome ambient construction site noise while maintaining comfortable listening levels for users. The sound system 122 may include a therapeutic audio content library stored in digital memory, comprising (but not limited to) guided meditation sessions, nature sounds, calming music, stress reduction audio programs, binaural beat audio programs at selectable beat frequencies, and/or any other therapeutic audio content.

A visual display system 124 may be configured to present synchronized relaxation imagery within the booth 102. The display system 124 may comprise high-resolution screens, projection systems, and/or the like, positioned to provide optimal viewing angles for seated users. One or more display controllers may synchronize visual content with audio programming to create coordinated therapeutic experiences. The visual system 124 may include libraries of relaxation imagery such as, but not limited to, natural landscapes, flowing water, calming abstract patterns, and/or any other visual imagery designed to reduce stress and/or promote mental wellness.

A haptic transducer array 127 may be disposed within the booth 102 to deliver vibrotactile stimulation to a user during a therapy session. The haptic transducer array 127 may comprise a plurality of vibrotactile actuators positioned within the seating element 114 and along interior wall surfaces of the booth 102 to provide full-body vibrotactile coverage. The actuators may be driven by a haptic controller communicatively coupled to the controller 152, which may coordinate haptic output timing and intensity with the therapeutic audio content delivered through the sound system 122 and the synchronized visual content displayed through the visual display system 124. The haptic transducer array 127 may deliver vibrotactile stimulation at frequencies and amplitudes selected based on the determined stress indicators, with lower-frequency, lower-amplitude stimulation patterns selected for users presenting with elevated stress profiles to promote parasympathetic nervous system activation. The haptic transducer array 127 may operate independently of or in coordination with the ambient lighting system 129 and the sound system 122 to produce a fully coordinated, synchronized multi-modal therapeutic output across audio, visual, lighting, and vibrotactile channels simultaneously.

In some embodiments, construction-specific therapeutic content may be integrated into the audio and visual libraries to address occupational stressors unique to construction environments. The content may include guided relaxation sessions that acknowledge construction work challenges, stress management techniques tailored to physical labor demands, and mindfulness exercises designed for workers wearing personal protective equipment. Construction-themed imagery may present positive construction environments, completed projects, and safe work scenarios to reinforce positive associations with the work environment.

Multi-sensory therapy delivery coordination system 126 may synchronize audio, visual, and environmental control systems to create immersive therapeutic experiences. The coordination system 126 may adjust lighting levels, temperature, and air circulation in conjunction with audio and visual content to enhance therapeutic effectiveness. The coordination system 126 may incorporate timing controllers to sequence different therapeutic modalities throughout user sessions to maintain engagement and optimize stress reduction outcomes.

In some embodiments, noise-cancellation hardware 128 may provide environmental isolation from construction site noise and distractions. Noise-cancellation hardware 128 may include an active noise cancellation system using microphones and speakers to generate sound waves that cancel ambient noise frequencies. Additionally or alternatively, the noise-cancellation hardware 128 may include passive acoustic isolation provided through sound-absorbing materials integrated into booth walls and ceiling surfaces. The noise-cancellation hardware 128 may enable users to focus on therapeutic content without interference from external construction activities, equipment operation, or environmental noise.

### C. A Biometric Monitoring System

The biometric monitoring system 130 may comprise contactless sensing arrays positioned within the booth 102 to collect physiological data from users without requiring physical contact. The system 130 may be configured to function effectively while users wear personal protective equipment commonly required at construction sites, including hard hats, safety glasses, and work clothing.

Baseline stress profile establishment protocols may collect physiological measurements during initial user registration sessions when users are in documented relaxed states, such as during pre-shift periods or break times. Multi-session baseline collection may gather measurements across multiple time periods and environmental conditions to establish robust individual normal ranges that account for circadian rhythms, caffeine consumption, and work schedule variations. Statistical analysis algorithms may calculate personalized threshold boundaries using percentile-based methods that define normal (0-25th percentile), elevated (25th-75th percentile), and critical (>75th percentile) stress ranges for each user.

Baseline profile updates may occur automatically through continuous learning algorithms that gradually adjust normal ranges based on long-term physiological trends while maintaining sensitivity to acute stress episodes. Seasonal adjustment protocols may account for environmental factors such as temperature extremes and daylight variations that affect baseline physiological parameters in construction environments. The system may implement baseline validation procedures that identify and exclude measurements collected during illness, medication changes, or other confounding factors that could compromise profile accuracy.

The biometric monitoring system 130 may implement specialized sensor configurations designed to accommodate standard construction personal protective equipment without requiring removal or modification. Hard hat compatibility features may include sensor positioning that avoids interference with hard hat suspension systems and chin straps while maintaining clear line-of-sight for facial thermal imaging. Safety glasses accommodation protocols may utilize infrared wavelengths that penetrate standard safety lens materials and employ multiple sensor angles to ensure reliable physiological monitoring despite eyewear obstruction.

Protective clothing penetration capabilities may enable heart rate and respiration monitoring through high-visibility vests, work shirts, and light jackets using millimeter-wave radar technology that operates effectively through fabric layers. Glove compatibility systems may eliminate requirements for hand-based biometric measurements, relying instead on facial and torso monitoring techniques that remain effective when users wear work gloves or protective hand coverings. The system may implement PPE detection algorithms that automatically adjust sensor sensitivity and measurement parameters based on detected protective equipment to maintain measurement accuracy across varying PPE configurations.

The contactless biometric monitoring subsystem may be configured such that the thermal imaging sensor, the optical heart rate sensor, and the motion-based respiration sensor each operate simultaneously without physical contact with the user and without requiring removal of any personal protective equipment by the user. The thermal imaging sensor may collect facial temperature data through infrared detection that is unaffected by the presence of hard hats, safety glasses, or high-visibility vests. The optical heart rate sensor may detect skin color variations in exposed facial regions using remote photoplethysmography techniques that do not require removal of gloves, headwear, or protective eyewear. The motion-based respiration sensor may detect chest wall displacement through millimeter-wave radar signals that penetrate standard work clothing, eliminating any requirement for the user to remove or adjust protective outerwear during physiological monitoring. The simultaneous, PPE-compatible operation of all three sensor types may enable continuous, uninterrupted physiological monitoring throughout the therapy session without imposing any additional burden on the user beyond simply being seated within the booth.

The biometric monitoring system 130 may include one or more thermal imaging cameras 132. The one or more cameras 132 may be positioned within the booth interior to perform physiological assessment through infrared detection. The cameras 132 may detect minute temperature variations in facial regions that correlate with heart rate, respiration patterns, and/or stress responses. Image processing algorithms may analyze thermal data to extract physiological parameters without requiring direct skin contact or removal of protective equipment. The thermal cameras 132 may operate in wavelength ranges that penetrate typical construction safety equipment while maintaining user privacy.

The thermal imaging sensors may comprise uncooled microbolometer arrays configured to detect infrared radiation in the 8-14 micrometer wavelength range corresponding to human body heat signatures. Thermal processing algorithms may analyze temperature distribution patterns across facial regions to identify stress-related vasodilation and vasoconstriction responses. Calibration protocols may automatically adjust thermal sensitivity based on ambient temperature conditions and user baseline measurements collected during initial booth entry. Signal processing components may apply noise reduction filters and temporal averaging to eliminate environmental thermal interference while preserving physiological temperature variations indicative of stress responses.

Heart rate sensors 134 may be integrated into the biometric monitoring system 130 to provide cardiovascular monitoring capabilities. The sensors 134 may utilize optical detection methods to identify pulse patterns through remote photoplethysmography techniques. Signal processing circuits may filter and analyze optical data to determine heart rate variability and cardiac rhythm patterns that indicate stress levels. The heart rate sensors 134 may function through safety glasses and face shields commonly worn by construction workers.

The optical heart rate sensors may utilize photoplethysmography (PPG) technology with dual-wavelength LED illumination at 525 nm and 940 nm to penetrate varying skin tones and detect blood volume changes in facial capillaries. Heart rate variability algorithms may analyze inter-beat intervals to calculate time-domain metrics including RMSSD and frequency-domain metrics including LF/HF ratios that correlate with autonomic nervous system activity. Motion artifact rejection systems may employ adaptive filtering and signal quality assessment to maintain accurate heart rate detection despite user movement or construction site vibrations.

Respiration sensors 136 may monitor breathing patterns to assess user stress and relaxation states. The sensors 136 may utilize thermal detection of exhaled air, optical monitoring of chest movement, and/or other techniques known in the art to determine respiration rate and depth. Breathing pattern analysis algorithms may identify irregular breathing associated with anxiety or stress conditions. The respiration monitoring may accommodate various seating positions and protective clothing configurations.

The motion-based respiration sensors may utilize millimeter-wave radar technology operating at 60 GHz to detect chest wall displacement patterns without requiring direct contact or removal of protective equipment. Respiration analysis algorithms may process displacement waveforms to extract breathing rate, depth variations, and irregularity patterns associated with stress responses. Breathing pattern classification systems may identify shallow breathing, breath holding, and hyperventilation episodes that indicate elevated stress levels requiring therapeutic intervention.

Motion detection arrays 138 may monitor user movement and posture within the booth 102. The arrays 138 may include multiple sensors positioned to detect fidgeting, restlessness, or other movement patterns that indicate stress or discomfort. Position and/or movement analysis systems may assess user positioning and movement, and provide feedback for optimal therapeutic positioning. The motion detection may distinguish between normal therapeutic relaxation movements and stress-related behaviors.

Optical sensing arrays 140 may provide additional physiological monitoring through advanced imaging techniques. The arrays 140 may detect subtle color changes in facial regions that correlate with blood flow and oxygenation levels. Machine learning algorithms may process optical data to identify stress indicators and/or emotional states. The optical systems 140 may operate in lighting conditions suitable for therapeutic environments while maintaining measurement accuracy.

Real-time vital sign collection systems 142 may continuously gather physiological data throughout user sessions. Data fusion algorithms may combine inputs from multiple sensor types to provide comprehensive physiological assessment. The collection systems 142 may maintain data sampling rates sufficient to detect rapid changes in stress indicators during therapeutic interventions.

Stress indicator determination systems 144 may analyze collected physiological data to identify user stress levels and emotional states. Pattern recognition algorithms may compare current measurements to baseline values and established stress response patterns. The determination systems 144 may provide real-time feedback to the controller for adaptive therapy content selection and session management.

### D. An Adaptive Control System

The adaptive control system 150 may comprise a central controller configured to receive and process physiological data from the biometric monitoring system 130 and coordinate therapeutic interventions through the therapy delivery subsystem 120. The control system 150 may provide autonomous operation while maintaining user privacy and data security throughout therapeutic sessions.

A controller 152 may be communicatively coupled to the biometric monitoring system 130 to receive physiological data in real-time or near real-time. The controller 152 may comprise processing hardware and/or software configured to handle multiple data streams simultaneously from thermal cameras, heart rate sensors, respiration monitors, and motion detection arrays. Data acquisition interfaces may convert analog sensor signals to digital formats suitable for processing and analysis. The controller 152 may maintain continuous data collection throughout user sessions to enable responsive therapeutic adjustments.

Stress indicator analysis algorithms 154 may process physiological data to determine user stress levels and emotional states. The algorithms 154 may compare current physiological measurements to baseline values established during initial session periods. Pattern recognition systems may identify stress response signatures including elevated heart rate, irregular breathing patterns, increased movement, and thermal variations. Machine learning components may adapt stress detection sensitivity based on individual user responses and therapeutic outcomes.

The machine learning components may implement ensemble neural network architectures combining convolutional neural networks for physiological signal pattern recognition with recurrent neural networks for temporal stress progression modeling. Training data acquisition systems may utilize federated learning protocols that enable model improvement across multiple booth installations while maintaining individual user privacy through differential privacy techniques. Model update mechanisms may implement incremental learning algorithms that adapt therapeutic recommendations based on user response feedback without requiring complete model retraining.

Privacy-preserving analytics components may utilize homomorphic encryption techniques that enable aggregate trend analysis across user populations while preventing individual data reconstruction. Local model inference engines may process user data entirely within the booth controller to minimize data transmission requirements and reduce privacy exposure risks. The machine learning system may implement model validation protocols that continuously assess prediction accuracy and automatically flag degraded performance requiring model updates or recalibration.

In some embodiments, the data processing subsystem may comprise or be communicatively coupled to a machine learning processor configured to analyze anonymized aggregate session data from a plurality of users across a plurality of therapy sessions. The machine learning processor may access anonymized session records stored by the anonymized biometric data storage subsystem 202 and apply supervised and unsupervised learning algorithms to identify correlations between initial stress indicator profiles, therapeutic content selections, and measurable stress reduction outcomes achieved across the user population. The machine learning processor may operate as a component of the data processing subsystem or as a dedicated processing unit communicatively coupled to the data processing subsystem through the controller 152, with the machine learning processor providing updated therapeutic content selection parameters to the therapeutic content selection systems 156 for application in subsequent therapy sessions.

The controller 152 may implement multi-parameter stress indicator calculation algorithms 154 that integrate physiological measurements into composite stress metrics suitable for therapeutic content selection. Weighted fusion algorithms may combine heart rate variability scores, thermal stress indicators, and respiration irregularity measures using machine learning-derived coefficients that account for individual physiological variations. Baseline deviation calculations may compare real-time measurements against established user-specific normal ranges to generate normalized stress scores ranging from 0-100, where values above 70 indicate elevated stress requiring immediate therapeutic intervention.

Temporal stress trend analysis may track stress indicator changes over 30-second intervals to identify acute stress episodes versus chronic elevated stress conditions. Adaptive threshold systems may automatically adjust stress classification boundaries based on user response patterns and therapeutic effectiveness measurements collected during previous sessions. The controller may implement safety override protocols 176 that immediately flag stress indicators exceeding critical thresholds (>90) for emergency alert generation regardless of user therapeutic preferences or session settings.

Therapeutic content selection systems 156 may choose appropriate audio and visual content based on biometric inputs and stress indicators. Content libraries may be organized by therapeutic effectiveness for different stress levels and user conditions. Selection algorithms may match current user physiological states to content categories optimized for stress reduction. The selection systems 156 may consider session duration, previous content effectiveness, and user response patterns when determining optimal therapeutic programming.

Therapeutic content selection systems 156 may maintain a comprehensive therapeutic content library comprising guided meditation sessions, breathing exercises, progressive muscle relaxation protocols, and cognitive behavioral therapy techniques specifically adapted for construction worker stress patterns. Content selection algorithms may analyze real-time stress indicators to automatically choose appropriate therapeutic interventions, with high-stress conditions triggering immediate calming content and moderate stress levels enabling longer-duration mindfulness sessions.

Adaptive content delivery systems may modify therapeutic session intensity, duration, and complexity based on user physiological responses measured during content delivery. Construction-themed therapeutic content may include guided visualizations of successful project completion, safety-focused mindfulness exercises, and stress management techniques specifically addressing construction industry challenges such as weather delays, safety concerns, and physical demands. Content effectiveness tracking may monitor user physiological improvements during each therapeutic modality to optimize future content selection and enable evidence-based therapy personalization.

Real-time therapy adjustment systems 158 may modify therapeutic content during user sessions based on ongoing physiological monitoring. Dynamic content controllers may adjust audio volume, visual brightness, session pacing, and/or environmental controls in response to changing stress indicators. Feedback loops may continuously evaluate therapeutic effectiveness and modify interventions to optimize stress reduction outcomes. The adjustment systems 158 may transition between different therapeutic modalities seamlessly to maintain user engagement and therapeutic benefit.

In some embodiments, the haptic transducer array 127 may be configured to deliver a bilateral alternating stimulation pattern in which a left-side transducer and a right-side transducer are activated in alternating sequence. The controller 152 may drive the left-side transducer and the right-side transducer in an alternating bilateral pattern synchronized with the therapeutic audio content delivered through the sound system 122. The alternating bilateral stimulation pattern may comprise activation of the left-side transducer for a first predetermined interval followed by activation of the right-side transducer for a second predetermined interval, with the cycle repeating at a rate selected based on the determined stress indicators. In some embodiments, the alternating rate may be set between approximately one and four cycles per second, with lower rates selected for users presenting with higher stress indicator scores to promote deeper relaxation responses. The bilateral alternating pattern may be coordinated with binaural beat audio content delivered through the sound system 122 such that the left-right alternation of haptic stimulation corresponds to the left-right audio channel alternation of the binaural beat signal, reinforcing the neurophysiological entrainment effect through simultaneous auditory and vibrotactile bilateral stimulation. The controller 152 may dynamically adjust the alternating rate and stimulation amplitude in real time based on changes in the physiological data monitored during the therapy session, increasing the rate and reducing the amplitude as stress indicators decrease toward baseline levels.

In some embodiments, the bilateral alternating stimulation pattern may be synchronized with therapeutic audio content other than binaural beat audio, including nature soundscapes, guided meditation programs, and breathing exercise audio content. When synchronized with a nature soundscape, the bilateral alternating pattern may be timed to coincide with rhythmic audio elements within the soundscape, such as wave sounds or wind patterns, with the left-side transducer activating in correspondence with audio elements presented through the left audio channel and the right-side transducer activating in correspondence with audio elements presented through the right audio channel. When synchronized with a guided breathing exercise, the bilateral alternating pattern may transition from left-side to right-side activation at a rate corresponding to the guided inhalation and exhalation cycle, providing a vibrotactile cue that reinforces the audio breathing guidance. The controller 152 may determine the synchronization timing for the bilateral alternating pattern based on the type and tempo of the therapeutic audio content currently being delivered, applying content-type-specific synchronization algorithms to maintain coherent coordination between the haptic and audio output channels across all therapeutic content types in the content library.

In some embodiments, the data processing subsystem may comprise or be communicatively coupled to a haptic controller configured to drive the left-side transducer and the right-side transducer of the haptic transducer array 127 in the alternating bilateral stimulation pattern. The data processing subsystem may coordinate the bilateral alternating stimulation pattern with the therapeutic audio content and the synchronized visual content delivered through the multi-sensory therapy delivery subsystem, such that the left-right alternation of haptic stimulation is synchronized with corresponding left-right audio channel transitions and visual content transitions to produce a fully coordinated bilateral multi-modal therapeutic output.

In some embodiments, the data processing subsystem may be referred to as or may comprise a controller configured to coordinate all therapeutic output channels, including the sound system, the visual display system, the haptic transducer array, and the ambient lighting system. The controller, as a component of or as implemented by the data processing subsystem, may be configured to activate the left-side transducer and the right-side transducer of the haptic transducer array in the alternating bilateral stimulation pattern described herein. References to “the controller” in the context of bilateral stimulation pattern activation, therapeutic content delivery coordination, and real-time adaptive adjustment may be understood to refer to the data processing subsystem or a processing component thereof, and the terms may be used interchangeably to describe the same functional capability implemented within the booth system architecture.

Secure data processing systems 160 may ensure privacy preservation throughout physiological monitoring and therapeutic delivery. Encryption algorithms may protect physiological data during collection, processing, and storage operations. Data anonymization systems may remove personally identifiable information while preserving therapeutic effectiveness data for system optimization. Access control mechanisms may restrict data access to authorized system functions and prevent unauthorized disclosure of user information.

Session management systems 162 may coordinate overall therapeutic session flow and user interaction. Timer functions may enforce session duration limits and manage booth availability for multiple users. User interface controllers may provide simple interaction methods for session initiation and termination. Emergency response systems may detect abnormal physiological readings and initiate appropriate safety protocols or alert procedures when necessary.

### E. An Automated Sanitization System

The automated sanitization system 170 may be configured to activate upon detection of booth vacancy following user sessions to ensure hygienic conditions for subsequent users. The system 170 may provide comprehensive disinfection of interior surfaces, air quality management, and contamination prevention suitable for shared-use applications in construction environments.

A vacancy detection system 172 may monitor booth occupancy to trigger sanitization cycles automatically. The vacancy detection system 172 may include one or more occupancy sensors (e.g., motion detectors, pressure sensors in seating areas, door position monitors to determine when users have exited the booth 102, etc.). Confirmation timers may ensure booth vacancy for predetermined periods before initiating sanitization procedures to prevent accidental exposure to disinfection systems. The detection systems 172 may distinguish between brief absences and session completion to avoid unnecessary sanitization cycles.

Ultraviolet light emitters 174 may provide surface disinfection throughout the booth interior. The Ultraviolet light emitters 174 may include UV-C light sources positioned at multiple locations within the booth 102 to ensure comprehensive coverage of interior surfaces (e.g., including walls, seating, control interfaces, and air circulation pathways). Reflective surfaces may be strategically positioned to direct ultraviolet radiation to shadowed areas and help to ensure uniform disinfection coverage. Safety interlocks may prevent UV operation when booth occupancy is detected to protect users from harmful radiation exposure.

Sanitizing mist dispensers 176 may provide comprehensive cleaning of surfaces and air spaces within the booth 102. The dispensers 176 may atomize disinfectant solutions to create fine droplets that settle on all interior surfaces. Distribution nozzles may be positioned to provide uniform mist coverage throughout the booth interior, including hard-to-reach areas and complex surface geometries. Ventilation coordination may control air circulation during mist application to optimize distribution and drying times.

Automated cleaning protocols may coordinate sanitization system operation to ensure effective disinfection between user sessions. Sequencing controllers may activate one or more of UV emitters 174, mist dispensers 176, and ventilation systems in predetermined sequences to maximize disinfection effectiveness. Timing systems may control exposure durations for UV radiation and/or mist contact times based on pathogen elimination requirements. Completion verification may monitor sanitization cycle progress and confirm successful disinfection before enabling booth availability for subsequent users.

Air filtration systems 178 may maintain clean air quality during and after sanitization cycles. The filtration systems 178 may comprise an air purification unit including a HEPA filter configured to remove airborne particulates from the booth interior during a sanitization cycle, capturing particulate matter, biological aerosols, and sanitization agent residues generated during UV irradiation and mist dispenser activation. The HEPA filter may activate at the commencement of each sanitization cycle and may continue operating at elevated capacity throughout the UV irradiation stage, the mist dispenser stage, and the post-sanitization recovery stage to ensure that airborne particulates and chemical residues are removed from the booth interior air before the booth is made available for the next user. Air quality sensors may monitor chemical concentrations and particulate levels to confirm that HEPA filtration has reduced airborne contaminant levels to safe thresholds before booth reoccupation is permitted. Ventilation controls may manage air exchange rates during sanitization and recovery periods to optimize both disinfection effectiveness and user safety.

Status indication systems may communicate sanitization cycle progress and booth availability to users and site personnel. Visual indicators may display sanitization status through colored lighting or display panels visible from outside the booth. Audio alerts may provide audible confirmation of sanitization completion and booth readiness. Remote monitoring interfaces may transmit sanitization status to site management systems for operational coordination and maintenance scheduling.

The sanitization system 170 may implement comprehensive safety interlock mechanisms to prevent user exposure to UV radiation and chemical sanitization agents during active sanitization cycles. UV safety interlocks may include motion sensors that immediately disable UV-C lamps upon detecting any movement within the booth, door position sensors that prevent UV activation when booth doors are not fully closed and locked, and emergency stop buttons accessible from both inside and outside the booth. UV exposure monitoring may track cumulative radiation levels and automatically limit sanitization duration to prevent material degradation while ensuring effective pathogen elimination.

Chemical mist safety systems may implement ventilation interlocks that ensure adequate air exchange before, during, and after mist sanitization cycles to prevent user exposure to sanitizing agents. Residue detection sensors may monitor air quality for chemical residues and prevent booth reoccupancy until safe levels are confirmed. The system may include visual and audible warning systems that clearly indicate active sanitization status through external warning lights and audio alerts, with multilingual safety messaging to accommodate diverse construction workforces.

### F. An Environmental Control System

The environmental control system 180 may comprise an integrated climate management subsystem configured to maintain optimal therapeutic conditions within the booth 102 while accommodating the challenging environmental conditions commonly encountered at construction sites. The system 180 may be communicatively coupled to the controller 152 to enable coordinated environmental adjustments based on user physiological data, external weather conditions, and therapeutic content requirements.

A temperature management subsystem 182 may be configured to regulate interior booth temperature through climate control mechanisms. The temperature management subsystem 182 may include heating elements positioned within booth walls and ceiling surfaces to provide uniform heat distribution during cold weather conditions. Cooling components may comprise compact air conditioning units or thermoelectric cooling systems configured to reduce interior temperatures during hot weather operations. Temperature sensors may monitor interior and exterior temperature conditions to enable automatic climate adjustments. The temperature management subsystem 182 may maintain interior temperatures within a predetermined therapeutic range, such as (but not limited to) between 68-76 degrees Fahrenheit, regardless of external construction site temperature variations.

The environmental control system 180 may be configured to maintain optimal moisture levels within the booth interior to enhance user comfort and protect sensitive electronic components. A humidity control subsystem 184 may include dehumidification components configured to remove excess moisture during humid weather conditions or when users generate elevated interior humidity levels. Humidification components may add moisture to booth air during dry conditions to prevent user discomfort and static electricity buildup. Humidity sensors may monitor interior moisture levels and provide feedback to the controller 152 for automatic humidity adjustments. The humidity control subsystem 184 may maintain relative humidity levels between 40-60 percent to optimize user comfort and equipment performance.

A ventilation management subsystem 186 may be configured to provide continuous air circulation and fresh air exchange within the booth 102. The ventilation management subsystem 186 may include intake fans positioned to draw external air into the booth interior while filtering incoming air to prevent dust and debris infiltration. Exhaust fans may be configured to remove stale air and optionally to maintain positive air pressure within the booth to prevent contamination from external construction activities. Air filtration components may comprise multi-stage filtration systems including particulate filters, activated carbon filters, HEPA filters, and/or the like to remove dust, chemical vapors, and airborne contaminants commonly present at construction sites. The ventilation management subsystem 186 may provide complete air exchange within the booth interior at predetermined intervals, such as every 3-5 minutes, to maintain air quality standards.

A noise cancellation and acoustic isolation subsystem may be configured to minimize external construction site noise intrusion and create optimal acoustic conditions for therapeutic content delivery. Active noise cancellation components may include microphone arrays positioned to detect external noise frequencies and speaker systems configured to generate inverse sound waves that cancel ambient construction noise. Passive acoustic isolation materials may be integrated into booth wall and ceiling structures to provide sound dampening and vibration isolation. The noise cancellation subsystem may reduce external noise levels by 25-40 decibels to enable clear therapeutic audio delivery without interference from construction equipment, vehicle traffic, or worker activities.

A lighting control subsystem 188 may be configured to provide therapeutic ambient lighting that functions as an active therapeutic output channel coordinated with the sound system 122, the visual display system 124, and/or the haptic transducer array 127 to enhance relaxation and supports circadian rhythm regulation. The ambient lighting system may include LED light arrays positioned throughout the booth interior and configured to deliver chromatic and intensity-variable light output synchronized with the therapeutic audio content and the relaxation imagery. In some embodiments, the ambient lighting system may further provide soft, adjustable illumination within the booth. Color temperature control systems may adjust lighting color from warm tones during relaxation sessions to cooler tones during alertness-promoting activities. Brightness control mechanisms may automatically adjust lighting intensity based on external daylight conditions, user preferences, and/or therapeutic content requirements. The lighting control subsystem 188 may synchronize with the visual display system 124 and audio content to create coordinated multi-sensory therapeutic experiences.

An air quality monitoring and filtration subsystem may be configured to continuously assess and maintain optimal air quality within the booth interior. Air quality sensors may monitor carbon dioxide levels, volatile organic compounds, particulate matter concentrations, and other air quality parameters. Advanced filtration systems may include activated carbon filters for chemical vapor removal, HEPA filters for particulate removal, and UV sterilization components for pathogen elimination. Air quality feedback systems may provide real-time air quality data to the controller to enable automatic filtration adjustments and ventilation modifications. The air quality monitoring subsystem may maintain interior air quality standards that exceed outdoor construction site conditions and meet or exceed indoor air quality guidelines for occupied spaces.

The environmental control system 180 may implement closed-loop feedback control algorithms that continuously adjust temperature, humidity, lighting, and air quality based on real-time user physiological responses and therapeutic content requirements. Biometric-responsive climate control may automatically reduce ambient temperature when thermal sensors detect elevated stress-related body heat and increase humidity levels when respiration monitoring indicates mouth breathing associated with anxiety responses. Therapeutic content synchronization may coordinate environmental adjustments with audio-visual content delivery, dimming lights during meditation sessions and adjusting color temperature to complement guided visualization exercises.

Predictive environmental control may utilize machine learning algorithms to anticipate optimal environmental conditions based on user stress patterns, time of day, and external weather conditions. Multi-zone environmental management may provide independent control of seating area, entry vestibule, and equipment zones to optimize user comfort while protecting sensitive electronic components. The system may implement environmental override protocols that prioritize user safety by maintaining minimum ventilation rates and safe temperature ranges even when therapeutic protocols might suggest different environmental settings.

### G. A User Interface and Session Management System

The user interface and session management system 190 may comprise an integrated control and monitoring subsystem configured to provide intuitive user interaction capabilities while ensuring safe and effective therapeutic session management. The system 190 may be communicatively coupled to the controller 152 to enable coordinated session control, user authentication, and emergency response protocols based on real-time biometric monitoring and user interaction patterns.

A presence detection subsystem 192 may be configured to automatically initiate session protocols upon detecting user entry into the booth 102. The presence detection subsystem 192 may include motion sensors positioned at booth entry points to detect user approach and entry movements. Proximity sensors may be configured to distinguish between casual proximity and intentional booth entry to prevent false session initiations. Weight sensors integrated into booth flooring may confirm user presence and provide additional verification of booth occupancy. The presence detection subsystem 192 may automatically activate booth systems, initiate user interface displays, and prepare therapeutic systems for immediate session commencement upon confirmed user presence.

A user authentication interface 194 may be configured to provide secure user identification and personalized session configuration capabilities. Biometric authentication components may include facial recognition cameras and/or fingerprint scanners configured to identify authorized users and retrieve personalized therapeutic preferences. Access card readers may accept employee identification cards or proximity badges for rapid user authentication in workplace environments. Personal identification number input interfaces may provide backup authentication methods when biometric systems are unavailable or compromised. The user authentication interface 194 may retrieve user-specific therapeutic profiles, session history data, and medical contraindications to customize therapeutic content delivery for individual users.

In some embodiments, the user authentication interface 194 may be configured to control booth access using a non-personally-identifying credential that permits user authentication without collecting or processing personally identifiable information. Non-personally-identifying credentials may include anonymous access tokens, randomly assigned numeric codes distributed to authorized workers without association to individual identity records, proximity badges encoded with randomized identifiers that cannot be traced to specific individuals without access to a separately maintained access control registry, and single-use session codes generated by a site safety officer and distributed without recording the recipient's identity. The non-personally-identifying credential may be validated by the user authentication interface 194 to confirm that the presenting user is an authorized worker without establishing or recording the user's individual identity within the booth system. In some embodiments, the non-personally-identifying credential may be a numeric PIN selected by the user and not stored in association with any personally identifiable information, such that the booth system records only that a valid credential was presented, without recording which specific individual presented it. This approach may enable equitable access control and session logging for workforce wellness analytics purposes while maintaining the anonymity protections required for sensitive occupational health monitoring applications.

A session time limit enforcement subsystem 196 may be configured to manage therapeutic session duration and ensure equitable booth access among multiple users. Session timer components may track elapsed session time and provide visual and auditory countdown notifications to users approaching session time limits. Automatic session termination mechanisms may gracefully conclude therapeutic content delivery and initiate booth exit procedures when predetermined time limits are reached. Session extension interfaces may allow authorized users to request additional session time when booth availability permits extended use. The session time limit enforcement subsystem 196 may maintain session logs for workplace productivity monitoring and ensure therapeutic resources remain available to all authorized users throughout work shifts.

In some embodiments, the session time limit enforcement subsystem 196 may be configured to support short-duration therapy sessions of between two minutes and fifteen minutes, enabling effective stress reduction within high-ambient-noise occupational deployment environments without requiring workers to leave the worksite for extended periods. Short-duration session protocols may be optimized to deliver the highest-impact therapeutic content within the available session window, with the controller 152 selecting content sequences that achieve measurable reductions in stress indicators within compressed timeframes. The portable booth may be configured to attenuate external ambient noise to a level sufficient to permit delivery of therapeutic audio content at a therapeutic efficacy level within high-ambient-noise deployment environments, including construction sites, industrial facilities, and manufacturing floors where ambient noise levels may exceed 85 decibels. In one example embodiment, a two-minute rapid stress reduction protocol may deliver an immediate high-intensity calming audio sequence paired with synchronized visual content and bilateral haptic stimulation at a rate calibrated to rapidly engage the parasympathetic nervous system, with the controller 152 selecting this protocol automatically when session time limits are set below five minutes and the user's initial stress indicator score exceeds a predetermined threshold. In another example embodiment, a fifteen-minute extended session protocol may deliver a progressive multi-phase therapeutic sequence transitioning from initial stress reduction through sustained relaxation to a gentle re-alerting phase, with the controller 152 coordinating all four sensory output channels throughout each phase transition to maintain therapeutic coherence across the full session duration.

In some embodiments, a therapeutic efficacy level for audio delivery may correspond to a signal-to-noise ratio within the booth interior sufficient to permit clear perception of therapeutic audio content by a user without requiring audio playback levels that could cause auditory discomfort or fatigue. The noise cancellation and acoustic isolation subsystem may reduce external ambient noise levels by between 25 and 40 decibels through the combined effect of passive acoustic isolation materials integrated into booth wall and ceiling structures and active noise cancellation components, such that in a high-ambient-noise deployment environment where external ambient noise levels may reach or exceed 85 decibels, the residual noise level within the booth interior may be reduced to a range compatible with therapeutic audio delivery at comfortable listening levels. In some embodiments, the controller 152 may monitor interior noise levels through microphones positioned within the booth interior and may automatically adjust therapeutic audio content playback levels to maintain a predetermined signal-to-noise ratio at the user's listening position throughout the therapy session.

An emergency alert system may be configured to detect abnormal biometric readings and initiate appropriate emergency response protocols. Biometric threshold monitoring components may continuously compare real-time physiological data against predetermined normal ranges and identify potentially dangerous deviations. Emergency notification interfaces may automatically contact designated emergency contacts, supervisors, or medical personnel when critical biometric anomalies are detected. Manual emergency activation controls may provide user-accessible panic buttons for immediate emergency assistance requests. The emergency alert system may override normal session protocols to prioritize user safety and ensure rapid emergency response when medical intervention may be required.

The emergency alert system may implement multi-tier alert criteria based on specific physiological threshold violations and escalation timeframes. Critical alert thresholds may include heart rate exceeding 180 BPM for more than 60 seconds, heart rate variability indicating severe autonomic dysfunction, core body temperature variations exceeding 2 °C from baseline, or respiration rates below 8 or above 30 breaths per minute. Moderate alert conditions may trigger when stress indicators exceed 85 for more than 5 minutes or when multiple physiological parameters simultaneously exceed normal ranges.

Emergency escalation protocols may automatically contact designated emergency contacts within 30 seconds of critical threshold detection, transmit encrypted health summaries to emergency medical services, and activate booth emergency lighting and audible alerts to attract immediate assistance. Manual override capabilities may allow users to cancel false alarms within 15 seconds while maintaining alert logs for safety compliance documentation. The system may implement emergency unlock protocols that automatically release booth door locks and activate emergency ventilation during critical medical events to ensure rapid emergency responder access.

A user-friendly controls and feedback system 198 may be configured to provide intuitive therapeutic session control and real-time user feedback capabilities. Touchscreen control interfaces may display therapeutic content options, session progress indicators, and environmental control adjustments in simplified, icon-based formats suitable for users wearing work gloves or protective equipment. Voice control systems may accept spoken commands for hands-free session control and therapeutic content selection. Haptic feedback components may provide tactile confirmation of user inputs and system responses through vibration patterns integrated into control surfaces. The user-friendly controls and feedback system subsystem may adapt interface complexity based on user experience levels and provide guided tutorials for first-time users to ensure effective therapeutic system utilization.

### H. A Data Management and Analytics Subsystem

The data management and analytics system 200 may comprise an integrated information processing and storage subsystem configured to collect, analyze, and securely manage therapeutic session data while maintaining strict privacy protections and enabling evidence-based therapy optimization. The system 200 may be communicatively coupled to all booth subsystems through the controller 152 to enable comprehensive data collection, real-time analytics processing, and secure data transmission to authorized external systems.

An anonymized biometric data storage subsystem 202 may be configured to securely collect and store physiological monitoring data while removing personally identifiable information to protect user privacy. Data anonymization components 204 may automatically strip personal identifiers from biometric readings and replace them with randomized session tokens that cannot be traced to individual users. Encrypted storage systems 206 may utilize advanced encryption protocols to protect stored biometric data from unauthorized access while maintaining data integrity for analytical purposes. The anonymized biometric data storage subsystem 202 may maintain separate databases for raw physiological measurements and processed wellness metrics to enable comprehensive analysis while preventing individual user identification.

An encrypted session summary generation subsystem 208 may be configured to create comprehensive therapeutic session reports while maintaining data security and user confidentiality. Session data compilation components 210 may aggregate biometric readings, therapeutic content selections, environmental conditions, and user interaction patterns into standardized summary formats. Encryption protocols 212 may secure session summaries using industry-standard cryptographic methods before transmission or storage. Summary distribution systems 214 may automatically deliver encrypted session reports to authorized healthcare providers, supervisors, or occupational health systems based on predetermined access permissions. The encrypted session summary generation subsystem 208 may provide detailed therapeutic effectiveness metrics while ensuring sensitive health information remains protected throughout the data lifecycle.

Therapy effectiveness metrics may comprise quantitative measurements of physiological improvement achieved during a therapy session, including the percentage reduction in composite stress indicator score from session baseline to session end, the change in heart rate from session entry to session completion, the change in heart rate variability from session entry to session completion, the change in respiration rate from session entry to session completion, and the duration of time during which the user's composite stress indicator score remained below a predetermined elevated stress threshold. In some embodiments, therapy effectiveness metrics may further include a content effectiveness score for each therapeutic content item delivered during the session, computed as the rate of stress indicator reduction observed during delivery of that content item relative to the rate observed during delivery of other content items in the same session. The therapy effectiveness metrics may be computed by the session effectiveness metrics module at session completion, packaged with the anonymized session identifier and session metadata, encrypted using industry-standard cryptographic protocols, and included in the encrypted session summary report for transmission to the external occupational health monitoring system.

A machine learning therapy personalization subsystem 216 may be configured to analyze user response patterns and optimize therapeutic content delivery for individual physiological profiles and stress management needs. Adaptive algorithm components 218 may process historical biometric data to identify optimal therapeutic interventions for specific stress patterns, fatigue levels, and physiological conditions. Personalization engines 220 may automatically adjust therapeutic content selection, session duration, and environmental parameters based on machine learning predictions of user response effectiveness. Continuous learning systems 222 may refine therapeutic recommendations based on real-time biometric feedback and session outcome measurements. The machine learning therapy personalization subsystem 216 may improve therapeutic effectiveness over time while adapting to changing user needs and physiological conditions.

The machine learning processor may optimize therapeutic content selection parameters by maximizing measurable stress reduction outcomes across the user population, wherein measurable stress reduction outcomes may be quantified as the mean percentage reduction in composite stress indicator score from session baseline to session end across a plurality of sessions, the proportion of sessions achieving a composite stress indicator score reduction exceeding a predetermined threshold, or the mean rate of stress indicator reduction per unit time during therapeutic content delivery. The machine learning processor may treat these quantitative outcome measures as optimization targets and may update content selection parameters, content sequencing rules, and biometric-to-content mapping functions to increase the expected value of the measurable stress reduction outcome for users presenting with specific initial stress indicator profiles. Parameter updates may be validated against a held-out set of anonymized historical sessions before deployment to confirm that the updated parameters produce statistically significant improvements in measurable stress reduction outcomes relative to the prior parameter set.

An aggregate wellness trend analysis subsystem 224 may be configured to identify workplace wellness patterns and provide organizational health insights without compromising individual user privacy. Population analytics components 226 may analyze anonymized data across multiple users to identify trends in workplace stress levels, therapeutic effectiveness, and overall wellness improvements. Trend visualization systems 228 may generate graphical reports showing aggregate wellness metrics, usage patterns, and therapeutic outcome trends for management review. Predictive modeling components 230 may forecast potential workplace wellness issues based on historical data patterns and environmental factors. The aggregate wellness trend analysis subsystem 224 may enable evidence-based workplace wellness program optimization while maintaining complete user anonymity.

Workforce wellness metrics may comprise population-level quantitative measurements derived from anonymized aggregate session data collected across a plurality of users over a defined time period, including the mean composite stress indicator score across all sessions within the time period, the percentage of sessions in which a measurable reduction in composite stress indicator score was achieved, the distribution of initial stress indicator scores across the user population, the mean percentage reduction in stress indicators achieved per session across the user population, the frequency of booth utilization per shift period, and the correlation between booth utilization rates and aggregate stress indicator trends over time. Workforce wellness metrics may further include identification of peak stress periods correlated with specific work shifts, project phases, or environmental conditions, enabling site managers to identify systemic stressors and schedule therapeutic resources accordingly. All workforce wellness metrics may be computed from anonymized aggregate data using statistical aggregation thresholds that prevent identification of any individual user's data within the reported metrics, with differential privacy techniques applied to ensure that the addition or removal of any single user's session data does not materially alter the reported metric values.

Privacy-preserving data handling protocol subsystems 232 may be configured to ensure all data collection, processing, and transmission activities comply with applicable privacy regulations and organizational confidentiality requirements. Data minimization components 234 may collect only essential information required for therapeutic effectiveness while avoiding unnecessary personal data gathering. Access control systems 236 may restrict data access to authorized personnel based on role-based permissions and legitimate business needs. Data retention policies 238 may automatically purge stored information according to predetermined schedules to minimize privacy exposure risks. The privacy-preserving data handling protocol subsystems 232 may maintain comprehensive audit trails of all data access activities while ensuring user privacy rights are protected throughout all system operations.

Integration subsystems for external occupational health systems 240 may be configured to enable secure data sharing with authorized healthcare providers and workplace wellness programs while maintaining strict privacy protections. Health information exchange interfaces 242 may facilitate secure transmission of therapeutic session summaries to occupational health providers for comprehensive employee wellness monitoring. Workplace wellness platform connectors 244 may integrate therapeutic session data with existing employee health and safety management systems. Compliance reporting systems 246 may generate automated reports for occupational health and safety regulatory requirements while protecting individual user privacy. The integration subsystems for external occupational health systems 240 may enable comprehensive workplace wellness coordination while ensuring all data sharing activities comply with healthcare privacy regulations and organizational confidentiality policies.

### III. Platform Operation

Embodiments of the present disclosure provide a hardware and software platform operative by a set of methods and computer-readable media comprising instructions configured to operate the aforementioned modules and computing elements in accordance with the methods. The following depicts an example of at least one method of a plurality of methods that may be performed by at least one of the aforementioned modules. Various hardware components may be used at the various stages of operations disclosed with reference to each module.

For example, although methods may be described as being performed by a single computing device, it should be understood that, in some embodiments, different operations may be performed by different networked elements in operative communication with the computing device. For example, at least one computing device 400 may be employed in the performance of some or all of the stages disclosed with regard to the methods. Similarly, an apparatus may be employed in the performance of some or all of the stages of the methods. As such, the apparatus may comprise at least those architectural components found in computing device 400.

Furthermore, although the stages of the following example method are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in arrangements that differ from the ones described below. Moreover, various stages may be added or removed from the method without altering or departing from the fundamental scope of the depicted methods and systems disclosed herein.

### A. Therapeutic Intervention Method

Consistent with embodiments of the present disclosure, a method may be performed by at least one of the aforementioned modules. The method may be embodied as, for example, but not limited to, computer instructions, which, when executed, perform the method.

In operation, the system 100 may implement a comprehensive therapeutic intervention method that seamlessly integrates user detection, biometric monitoring, and personalized content delivery to provide on-demand mental health support for construction workers. The method may begin with automated user presence detection through the user interface and session management system 190, which may identify approaching workers and initiate booth preparation protocols including environmental conditioning and system activation. Upon user entry, biometric authentication components may verify user identity and retrieve personalized therapeutic profiles while contactless physiological monitoring sensors begin collecting baseline stress indicators, heart rate variability, and other wellness metrics.

The method may continue with real-time stress level analysis performed by the controller 152, which may process incoming biometric data against established baseline parameters and machine learning algorithms to determine optimal therapeutic interventions. Based on this analysis, the system may automatically select and deliver personalized therapeutic content through the audio-visual delivery system 124, while simultaneously adjusting environmental conditions through the environmental control system 180 to create optimal therapeutic ambiance. Throughout the session, the method may continuously monitor user physiological responses and dynamically adjust therapeutic content intensity, duration, and modality to maximize stress reduction effectiveness. Upon session completion, the method may implement automated sanitization protocols through the hygiene and sanitization system 170 while generating encrypted session summaries through the data management and analytics system 200, ensuring the booth is prepared for the next user while maintaining comprehensive wellness data for ongoing therapeutic optimization.

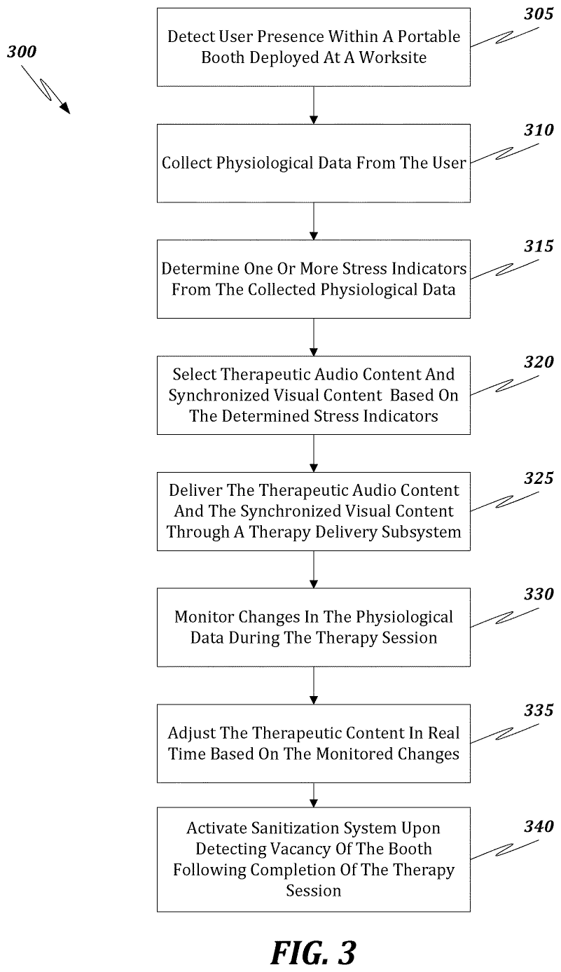

FIG. 3 is a flow chart setting forth the general stages involved in a method 300 consistent with an embodiment of the disclosure for providing therapeutic intervention using the portable therapy booth system 100. Method 300 may begin at stage 305 where presence of a user is detected within a portable booth deployed at a worksite. The presence detection may be performed using a combination of sensor modalities to ensure accurate and reliable determination of booth occupancy. In some embodiments, the presence detection subsystem 192 may employ motion detectors positioned at booth entry points to identify movement within the booth interior, proximity sensors configured to detect a user approaching and entering the booth, and weight sensors integrated into the booth flooring or seating element to confirm physical occupancy. The redundant, multi-modal detection approach may prevent false positives and false negatives in occupancy determination, ensuring that therapy sessions are initiated only upon confirmed user presence. In one example embodiment, a passive infrared motion detector positioned above the booth entry may detect the thermal signature of an approaching user, while a floor-mounted pressure sensor simultaneously confirms that the user's weight is present within the booth interior. The combination of these two independent signals may trigger session initiation protocols, including activation of interior lighting, audio-visual systems, and biometric sensor arrays. In another example embodiment, a radar-based presence detection system may be employed to detect user entry through the booth door, providing occupancy confirmation that is independent of ambient lighting conditions and effective regardless of whether the user is wearing personal protective equipment such as a hard hat or high-visibility vest.

Upon detecting user presence, the method 300 may continue to stage 310 where physiological data is collected from the user using a plurality of contactless biometric sensors positioned within the portable booth, wherein the physiological data is collected without physical contact with the user. The contactless biometric sensing array may begin data acquisition immediately upon confirmed user presence, enabling continuous physiological monitoring throughout the therapy session without requiring the user to remove personal protective equipment, attach wearable devices, or interact with any physical sensor interface. The contactless design may be particularly significant in construction and industrial worksite environments where workers routinely wear gloves, hard hats, safety glasses, and other protective gear that would otherwise preclude conventional contact-based biometric monitoring.

In some embodiments, the physiological data collection at stage 310 may comprise capturing thermal images of the user to detect facial temperature patterns. The thermal imaging camera 132 may be positioned within the booth interior at a location providing an unobstructed view of the user's facial region, particularly the forehead and periorbital areas where stress-associated temperature variations are most reliably detectable. The thermal imaging camera 132 may operate in the mid-wave or long-wave infrared spectrum, capturing thermal image frames at a rate sufficient to detect transient temperature changes associated with autonomic stress responses, including stress-related vasodilation and vasoconstriction patterns in facial skin regions. In one example embodiment, the thermal imaging camera 132 may capture thermal image frames at a rate of at least fifteen frames per second, enabling detection of dynamic temperature fluctuations that correlate with cardiovascular activity and emotional arousal states. The thermal imaging may function effectively in the variable ambient temperature conditions encountered at construction sites, with calibration algorithms compensating for environmental temperature variations to maintain measurement accuracy across a range of deployment conditions.

The physiological data collection at stage 310 may further comprise measuring heart rate by optically detecting skin color variations caused by blood volume changes. The heart rate sensors 134 may employ remote photoplethysmography techniques, detecting the subtle periodic changes in skin reflectance that occur as blood volume in superficial capillaries fluctuates with each cardiac cycle. Specialized optical sensors and illumination sources may be positioned within the booth to illuminate the user's facial skin region and detect the resulting reflectance variations. Signal processing algorithms may extract the cardiac pulse waveform from the optical reflectance signal, computing instantaneous heart rate and heart rate variability parameters from the extracted waveform. In one example embodiment, the optical heart rate sensor may utilize multiple wavelength bands, including green and near-infrared wavelengths, to enhance signal quality across users with varying skin tones and under varying ambient lighting conditions within the booth. The multi-wavelength approach may improve the reliability of heart rate extraction by selecting the wavelength band providing the highest signal-to-noise ratio for a given user's skin characteristics. In another example embodiment, the heart rate sensor may be configured to track heart rate variability in addition to instantaneous heart rate, computing time-domain metrics such as the root mean square of successive differences between consecutive inter-beat intervals, which may serve as a sensitive indicator of autonomic nervous system balance and stress state.

The physiological data collection at stage 310 may additionally comprise detecting respiration rate through non-contact sensing of chest movement. The respiration sensors 136 may employ one or more of radar-based micro-motion detection, optical flow analysis of video imagery, or depth-sensing technology to detect the subtle periodic displacement of the user's chest wall associated with respiratory cycles. In one example embodiment, a radar-based respiration sensor may emit low-power radio frequency signals that penetrate the user's clothing and detect the resulting micro-Doppler signatures of chest wall movement, enabling respiration rate measurement that is independent of the user's clothing configuration and effective even when the user is wearing bulky protective outerwear. In another example embodiment, a camera-based optical flow system may analyze sequential video frames to detect the periodic motion of the user's chest and abdomen associated with inhalation and exhalation cycles, computing respiration rate, breathing depth, and the regularity of the respiratory pattern. Irregular or shallow breathing patterns detected by the respiration sensors 136 may be identified as indicators of elevated stress or anxiety, contributing to the composite stress indicator profile generated at the subsequent stage of the method.

The method 300 may continue at stage 315 where one or more stress indicators are determined from the collected physiological data. The stress indicator determination systems 144 may process the raw physiological data streams received from the contactless biometric sensing array to extract quantitative physiological parameters and apply stress indicator determination algorithms to generate a composite stress indicator profile for the current user. The physiological parameters extracted may include instantaneous heart rate in beats per minute, heart rate variability metrics including root mean square of successive differences and frequency-domain power spectral components, respiration rate in breaths per minute, respiration depth and regularity indices, and facial skin temperature values and spatial distribution patterns across stress-sensitive facial regions.

The stress indicator determination algorithms may apply threshold-based classification, pattern recognition, and machine learning-based inference to the extracted physiological parameters to determine the user's current stress level and emotional state. In one example embodiment, the stress indicator determination algorithm may apply a multi-parameter composite scoring approach in which elevated heart rate above a predetermined threshold, reduced heart rate variability below a predetermined threshold, elevated respiration rate above a predetermined threshold, and elevated facial skin temperature in stress-associated regions each contribute weighted components to a composite stress indicator score. The composite score may be classified into discrete stress level categories, such as baseline or normal, mild stress, moderate stress, and severe stress, with each category mapped to corresponding therapeutic content selection parameters. In another example embodiment, the stress indicator determination algorithm may employ a trained machine learning classifier that receives the extracted physiological parameter vector as input and outputs a continuous stress indicator score or a probability distribution over stress level categories, with the classifier having been trained on anonymized aggregate session data from a plurality of prior users to improve classification accuracy across diverse physiological profiles.

At the start of each therapy session, the controller 152 may collect physiological data during an initial measurement window to establish a user-specific baseline stress profile. In one example embodiment, the baseline measurement window may comprise a period of approximately thirty to sixty seconds during which the user is seated within the booth and the contactless biometric sensing array collects physiological data prior to the commencement of therapeutic content delivery. The baseline stress profile established during this initial measurement window may represent the user's physiological state at session entry and may serve as a reference point for initial therapeutic content selection, real-time adaptive adjustment of therapeutic content during the session, and quantification of stress reduction achieved over the course of the session. In another example embodiment, for users who have completed prior therapy sessions within the system, the controller 152 may retrieve historical baseline data associated with the user's anonymized session identifier and incorporate historical physiological patterns into the baseline establishment process, enabling more accurate characterization of the user's typical physiological state and more precise detection of deviations from that baseline during the current session.

The method 300 may proceed at stage 320 where therapeutic audio content and synchronized visual content are selected based on the determined stress indicators. The therapeutic content selection systems 156 may query a therapeutic content library organized by stress level category, content type, and therapeutic modality to identify audio and visual content optimized for the user's current stress indicator profile. The content library may comprise a plurality of therapeutic audio content items including guided relaxation and mindfulness audio programs, nature soundscapes, instrumental music compositions, white noise and pink noise recordings, and binaural beat audio content at frequencies selected based on the determined stress indicators. The content library may further comprise a plurality of synchronized visual content items including natural landscape imagery, abstract calming animations, guided breathing visual cues, and biofeedback visualizations configured to display real-time stress indicator trends to the user.

The content selection at stage 320 may further comprise selecting haptic stimulation parameters for the haptic transducer array based on the determined stress indicators. Haptic stimulation parameters may include vibrotactile stimulation frequency, amplitude, and bilateral alternating rate, with lower frequencies and amplitudes selected for users presenting with elevated stress profiles to promote parasympathetic nervous system activation and higher frequencies and amplitudes selected for users presenting with mild stress profiles. In one example embodiment, for a user presenting with a severe stress indicator profile, the content selection system may select a low-frequency, low-amplitude bilateral alternating haptic stimulation pattern at a rate of approximately one cycle per second, coordinated with a theta-range binaural beat audio program and slow-paced relaxation imagery. In another example embodiment, for a user presenting with a mild stress indicator profile, the content selection system may select a moderate-frequency continuous vibrotactile pattern without bilateral alternation, coordinated with a nature soundscape audio program and natural landscape visual imagery.

In one example embodiment, for a user presenting with a severe stress indicator profile characterized by elevated heart rate, reduced heart rate variability, elevated respiration rate, and elevated facial skin temperature, the content selection system may select a guided relaxation audio program incorporating slow rhythmic audio elements designed to entrain the user's heart rate toward a lower target rate, paired with synchronized visual content displaying slowly expanding and contracting shapes that provide a visual respiratory guidance cue at a target breathing rate below the user's measured respiration rate. The combination of rhythmic audio entrainment and visual respiratory guidance may address the specific physiological manifestations of the user's elevated stress state by simultaneously targeting cardiovascular and respiratory stress indicators. In another example embodiment, for a user presenting with a mild stress indicator profile, the content selection system may select a nature soundscape audio program paired with synchronized natural landscape visual imagery, providing a less intensive therapeutic intervention appropriate for the user's lower stress level while still delivering meaningful stress reduction benefit.

The sound system 122 may be further configured to deliver binaural beat audio content at frequencies selected based on the determined stress indicators. Binaural beat audio is generated by delivering two audio tones of slightly different frequencies to the left and right ears respectively, producing a perceived beat frequency equal to the difference between the two tone frequencies. The perceived beat frequency may correspond to target brainwave frequency ranges associated with desired neurophysiological states, including alpha band frequencies in the range of approximately eight to twelve hertz associated with relaxed, calm states, and theta band frequencies in the range of approximately four to seven hertz associated with deeper meditative states. In one example embodiment, for a user presenting with a moderate stress indicator profile, the content selection system may select binaural beat audio content delivering a perceived beat frequency of approximately ten hertz, targeting the alpha brainwave frequency range associated with relaxed alertness, with the binaural beat content embedded within a nature soundscape audio program to provide a pleasant listening experience while delivering the neurophysiological entrainment effect. In another example embodiment, for a user presenting with a severe stress indicator profile, the content selection system may select binaural beat audio content delivering a perceived beat frequency of approximately six hertz, targeting the theta brainwave frequency range associated with deeper relaxation, with the frequency selection dynamically matched to the severity of the user's determined stress indicator profile.

The method 300 may continue at stage 325 where the therapeutic audio content is delivered through a sound system disposed within the portable booth and the synchronized visual content is displayed through a visual display system disposed within the portable booth. The sound system 122 may deliver the selected therapeutic audio content through speakers positioned at multiple locations within the booth interior to create an immersive audio environment. Audio amplifiers may drive the speakers with sufficient power to deliver therapeutic audio content at comfortable listening levels while overcoming residual ambient construction site noise that may penetrate the booth structure. The visual display system 124 may display the selected synchronized visual content on high-resolution screens or projection surfaces positioned to provide optimal viewing angles for a seated user within the booth interior. Display controllers may synchronize visual content playback timing with audio content playback to ensure that what the user sees and hears work together coherently rather than competing for attention.

In one example embodiment, the multi-sensory therapy delivery coordination system 126 may synchronize the audio content, visual content, haptic stimulation, and ambient lighting within the booth to create a fully integrated multi-sensory therapeutic experience. When the audio content transitions from an introductory nature soundscape to a guided breathing exercise, the visual content may simultaneously transition from a static natural landscape image to an animated breathing guidance visualization, the haptic transducer array may simultaneously shift from a continuous low-frequency vibrotactile pattern to a bilateral alternating pattern timed to the guided breathing cycle, and the ambient lighting may simultaneously shift from a neutral white illumination to a soft blue-green hue associated with calming effects, with all transitions occurring in coordinated sequence under the direction of the controller 152. In another example embodiment, the sound system 122 may deliver therapeutic audio content through a spatial audio configuration in which different audio elements are presented from different spatial positions within the booth, creating a three-dimensional audio environment that enhances the immersive quality of the therapeutic experience and more effectively draws the user's attention away from external construction site noise and distractions.

The method 300 may continue at stage 330 where changes in the physiological data are monitored during the therapy session. The real-time vital sign collection systems 142 may continuously gather physiological data from the contactless biometric sensing array throughout the duration of the therapy session, maintaining data sampling rates sufficient to detect rapid changes in stress indicators during therapeutic interventions. The controller 152 may receive continuous physiological data streams from the biometric monitoring system 130 and process the incoming data in real time to extract updated physiological parameter values and generate updated stress indicator assessments at regular intervals throughout the session. In one example embodiment, the controller 152 may update the composite stress indicator score at intervals of approximately five seconds throughout the therapy session, enabling detection of both rapid physiological changes and gradual trends in stress reduction over the course of the session. The continuous monitoring may establish trend lines for each measured physiological parameter, allowing the controller 152 to detect both sudden shifts in physiological state and gradual improvements in stress indicators that indicate the therapeutic content is producing the intended effect.

The method 300 may continue at stage 335 where the synchronized, coordinated multi-sensory therapeutic content may be adjusted in real time based on the monitored changes. The real-time therapy adjustment systems 158 may modify therapeutic content parameters during the user session based on ongoing physiological monitoring, implementing dynamic content adjustments that optimize stress reduction outcomes for the individual user's evolving physiological state. The feedback loops within the adaptive control system 150 may continuously evaluate therapeutic effectiveness by comparing the current stress indicator profile to the baseline stress profile and to the expected therapeutic trajectory, and may modify therapeutic interventions to optimize stress reduction outcomes when the current trajectory deviates from the expected path.

In one example embodiment, if the controller 152 detects that the user's heart rate is decreasing in response to the current therapeutic content but the user's respiration rate remains elevated and irregular, the real-time adjustment system may enhance the respiratory guidance elements within both the audio and visual content streams. The audio content may be adjusted to increase the prominence of slow, rhythmic breathing cues within the soundscape, while the visual content may be adjusted to display a more prominent animated breathing guidance visualization with a target breathing rate calibrated to guide the user toward a slower, more regular respiratory pattern. The adjustments may be implemented gradually through smooth content transitions that avoid disrupting the user's developing relaxation state. In another example embodiment, if the controller 152 detects that the user's composite stress indicator score is not decreasing at the expected rate after a predetermined period of therapeutic content delivery, the real-time adjustment system may transition the content selection to a higher-intensity relaxation modality, such as shifting from a nature soundscape to a guided meditation program with explicit relaxation instructions, or shifting the binaural beat frequency from an alpha-range target to a theta-range target to promote deeper relaxation. In yet another example embodiment, if the controller 152 detects that the user's physiological indicators are improving rapidly and the composite stress indicator score has decreased to a level consistent with a mild or baseline stress profile, the real-time adjustment system may transition the therapeutic content to a maintenance modality designed to sustain the achieved relaxation state for the remainder of the session rather than continuing to apply intensive stress reduction interventions.

In some embodiments, the real-time adjustment system may modify haptic transducer array output parameters in response to monitored changes in physiological data. In one example embodiment, if the controller 152 detects that the user's composite stress indicator score is decreasing steadily in response to the current therapeutic content, the real-time adjustment system may gradually reduce the amplitude of the bilateral alternating haptic stimulation pattern while simultaneously increasing the alternating rate from a low-stress-response rate toward a maintenance rate, providing a smooth transition that reflects the user's improving physiological state without abruptly terminating the haptic therapeutic input. In another example embodiment, if the controller 152 detects a sudden increase in the user's heart rate during a session, the real-time adjustment system may immediately reduce the haptic stimulation rate to the lowest configured rate and reduce stimulation amplitude to a minimal level, preventing the haptic output from contributing to physiological arousal while the audio and visual content transitions to a higher-intensity calming modality. The real-time adjustment system may similarly modify ambient lighting system output parameters in response to monitored physiological changes, shifting chromatic output toward warmer, lower-intensity tones when stress indicators increase and transitioning toward cooler, slightly brighter tones as stress indicators decrease toward baseline, with all lighting transitions implemented as gradual ramps rather than abrupt changes to avoid disrupting the user's developing relaxation state.

In some embodiments, the controller 152 may further be configured to detect an abnormal biometric reading from the contactless biometric sensing array and generate an emergency alert in response to the detected abnormal biometric reading. Abnormal biometric reading thresholds may include heart rate values exceeding or falling below clinically defined emergency thresholds, absence of detected respiration for a predetermined duration, or sudden loss of detected user presence combined with abnormal prior biometric readings. Upon detection of an abnormal reading, the controller 152 may generate an emergency alert transmitted to on-site safety personnel or emergency response systems via the secure communication module, and may simultaneously activate local audible or visual alarm indicators to attract attention to the booth location. In one example embodiment, if the thermal imaging camera 132 detects a sudden and sustained elevation in facial skin temperature combined with a simultaneous elevation in heart rate above a predetermined emergency threshold, the controller 152 may classify the combined biometric pattern as a potential acute health event and generate an emergency alert transmitted to the designated on-site safety officer via a push notification to a mobile device, while simultaneously activating an audible alarm within and outside the booth and initiating automatic door opening to facilitate rapid access by emergency responders.

The method 300 may conclude at stage 340 where an automated sanitization system within the portable booth is activated upon detecting vacancy of the portable booth following completion of the therapy session. The vacancy detection system 172 may monitor booth occupancy using multiple independent occupancy detection modalities to confirm that the user has exited the booth before initiating the sanitization cycle. In one example embodiment, vacancy confirmation may require concurrent agreement from at least two independent occupancy detection modalities, such as a passive infrared motion detector indicating absence of movement within the booth interior and a floor-mounted weight sensor indicating absence of user weight on the booth floor, with a confirmation timer requiring that both modalities indicate vacancy for a predetermined period of at least ten seconds before the sanitization cycle is initiated. The dual-modality confirmation approach may reduce the risk of false vacancy detection and prevent accidental activation of the sanitization system while a user remains within the booth.

Upon confirmed vacancy, the automated sanitization system 170 may execute a complete sanitization cycle comprising a predetermined sequence of disinfection operations. In one example embodiment, the sanitization cycle may comprise the following sequential stages: first, the air filtration systems 178 may activate at elevated capacity to begin removing airborne particulates and biological aerosols from the booth interior; second, the ultraviolet light emitters 174 may activate for a predetermined exposure duration, emitting UV-C radiation at wavelengths effective for inactivating bacteria, viruses, and other pathogens on interior surfaces, with reflective surfaces positioned to direct ultraviolet radiation to shadowed areas and ensure uniform disinfection coverage throughout the booth interior; third, the sanitizing mist dispensers 176 may activate to atomize a disinfectant solution and dispense a fine mist throughout the booth interior, with distribution nozzles positioned to achieve uniform surface coverage including hard-to-reach areas and complex surface geometries; fourth, the air filtration systems 178 may continue operating at elevated capacity to clear residual sanitizing mist from the booth interior air; and fifth, upon completion of the sanitization cycle, the controller 152 may log the sanitization cycle completion with a timestamp, update the booth status to indicate readiness for the next user, and update external status indicators to communicate booth availability to workers approaching the booth from the surrounding worksite. In another example embodiment, the sanitizing mist dispenser 176 may dispense a vaporized hydrogen peroxide solution formulated to achieve hospital-grade surface disinfection while remaining compatible with the antimicrobial interior surface materials of the booth and the sensitive electronic components of the biometric sensing array and audio-visual delivery systems. Safety interlocks within the sanitization control software may prevent activation of the ultraviolet light emitters 174 or the sanitizing mist dispensers 176 at any time when booth occupancy is detected by any of the occupancy detection modalities, ensuring that users are protected from exposure to disinfection agents and ultraviolet radiation throughout all operational states of the booth.

In some embodiments, the method 300 may further comprise storing anonymized biometric data and session information in encrypted local storage, generating an encrypted session summary report comprising anonymized biometric data and therapy effectiveness metrics, and transmitting the encrypted session summary report to an external occupational health monitoring system. The data anonymization and encryption module may automatically strip personal identifiers from biometric readings and replace them with randomized session tokens that cannot be traced to individual users, and may encrypt all anonymized data using industry-standard cryptographic protocols before committing the data to secure local storage within the booth's computing system. The encrypted session summary report may compile key data points from the therapy session, including the anonymized session identifier, session duration, baseline stress indicator values, final stress indicator values, percentage reduction in composite stress indicator score from baseline to session end, therapeutic content selections made during the session, and real-time adjustment events with associated physiological data. In one example embodiment, the secure communication module may transmit the encrypted session summary report to an external occupational health monitoring system via an encrypted wireless communication channel upon session completion, or may store the report in encrypted local storage for batch transmission when network connectivity becomes available, with verification mechanisms confirming successful receipt by the authorized external system and fallback procedures managing transmission failures.

In some embodiments, the method 300 may further comprise analyzing anonymized aggregate physiological data from a plurality of user sessions using a machine learning processor and generating workforce wellness metrics identifying stress trends across a plurality of users while maintaining individual user privacy. The machine learning therapy personalization subsystem 216 may access anonymized aggregate session data from a plurality of completed user sessions stored in encrypted local storage, analyze correlations between initial stress indicator profiles, therapeutic content selections made, and measurable stress reduction outcomes, and generate updated therapeutic content selection parameters that improve stress reduction outcomes across subsequent sessions. In one example embodiment, the machine learning processor may identify that users presenting with a specific combination of elevated heart rate and irregular respiration at session entry achieve significantly greater stress reduction when the initial therapeutic content selection includes a guided breathing exercise with explicit respiratory guidance cues compared to a nature soundscape without respiratory guidance, and may update the content selection parameters to increase the probability of selecting guided breathing content for users presenting with that specific biometric pattern. The aggregate wellness trend analysis subsystem 224 may further generate population-level workforce wellness analytics from the anonymized aggregate session data, identifying stress trends across the workforce, peak stress periods correlated with specific work shifts or project phases, and therapy effectiveness metrics at the population level, all formatted for transmission to the external occupational health monitoring system while maintaining complete individual user anonymity through statistical aggregation thresholds and differential privacy techniques.

In some embodiments, the method 300 may further comprise regulating temperature, humidity, and ventilation within the portable booth and activating active noise-cancellation hardware to attenuate external ambient noise within the portable booth interior. The environmental control system 180 may continuously monitor temperature, humidity, and air quality within the booth interior and regulate these parameters to maintain optimal therapeutic conditions throughout the user session. The temperature management subsystem 182 may maintain interior temperatures within a predetermined therapeutic range regardless of external construction site temperature variations, while the humidity control subsystem may maintain relative humidity levels within a range optimized for user comfort and equipment performance. The ventilation management subsystem 184 may provide continuous air circulation and fresh air exchange while filtering incoming air to remove dust, chemical vapors, and airborne contaminants commonly present at construction sites. The noise cancellation and acoustic isolation subsystem 186 may activate active noise cancellation components comprising microphone arrays positioned to detect external noise frequencies and speaker systems configured to generate inverse sound waves that destructively interfere with incoming external noise, reducing the transmission of external industrial noise into the booth interior by a sufficient margin to enable clear therapeutic audio delivery without interference from construction equipment, vehicle traffic, or worker activities. In one example embodiment, the active noise cancellation system may employ adaptive algorithms that continuously sample external ambient noise through microphones positioned on the exterior of the booth structure, analyze the frequency content and amplitude of the detected noise, and generate anti-phase signals through the booth's speaker system with timing and amplitude calibrated to achieve destructive interference with the incoming noise at the user's listening position within the booth interior. The adaptive algorithms may be specifically configured to target noise frequency signatures characteristic of construction site environments, including low-frequency machinery engine noise, mid-frequency impact sounds, and high-frequency pneumatic tool noise, providing effective noise attenuation across the frequency range most disruptive to therapeutic audio content delivery.

In some embodiments, the method 300 may further comprise establishing a baseline stress profile for the user based on physiological data collected at the start of the therapy session and comparing physiological data collected during the therapy session to the baseline stress profile to quantify a measurable reduction in stress indicators achieved during the session. The baseline establishment module may activate at session initiation following user presence detection and may collect physiological data during an initial measurement window of approximately thirty to sixty seconds, computing baseline stress indicator values from the initial measurement window data and storing the baseline profile for use by the real-time adjustment module and the session effectiveness metrics module. The session effectiveness metrics module may, at session completion, retrieve the baseline stress profile and the final session physiological data and compute quantitative session effectiveness metrics including the percentage reduction in heart rate from baseline to session end, the percentage reduction in composite stress indicator score from baseline to session end, the change in heart rate variability from baseline to session end, and the change in respiration rate from baseline to session end. In one example embodiment, the session effectiveness metrics may be presented to the user at session completion through a simplified display on the visual display system 124, showing a straightforward stress reduction indicator such as a percentage improvement in overall stress level, providing the user with objective feedback on the therapeutic benefit achieved during the session without exposing detailed biometric data. The session effectiveness metrics may also be packaged with the anonymized session data for inclusion in the encrypted session summary report transmitted to the external occupational health monitoring system, enabling population-level analysis of therapy effectiveness across the workforce.

### B. Example Therapeutic Session Implementation

In an exemplary therapeutic session implementation, the system 100 may deliver a standardized 20-minute mindfulness-based intervention designed to reduce occupational stress through controlled sensory reduction and guided interoceptive awareness training. Upon user authentication and booth entry, the environmental control system 180 may automatically configure the therapeutic environment by dimming ambient lighting to create a softly illuminated space, attenuating external construction site noise through active noise cancellation, minimizing visual distractions by deactivating non-essential display elements, and maintaining stable temperature conditions optimized for relaxation. The ergonomic seating system may adjust to provide comfortable postural support while the user settles into the therapeutic environment.

The multi-sensory therapy delivery subsystem 120 may initiate guided mindfulness content delivery through the sound system 122, the visual display system 124, the haptic transducer array 127, and the ambient lighting system 129, providing structured breathing cues that guide the user through controlled respiratory patterns designed to activate parasympathetic nervous system responses. Attentional anchoring instructions may direct the user's focus to specific physiological sensations, while prompts for interoceptive awareness may encourage conscious attention to internal bodily signals such as heartbeat, breathing rhythm, and muscle tension patterns. The biometric monitoring system 130 may continuously track physiological responses including heart rate variability, respiratory patterns, and stress indicators throughout the session, enabling the controller 152 to verify therapeutic effectiveness and ensure user safety. Upon session completion, the system may gradually restore normal environmental conditions while the data management and analytics system 200 processes session data to refine future therapeutic interventions based on measured physiological improvements and user response patterns.

### C. Deployment Scenarios and Use Cases

The system 100 may be deployed across diverse construction environments to address varying operational requirements and site-specific challenges through adaptable configuration and deployment strategies.

In urban high-rise construction project deployments, the system 100 may be configured to address unique stressors associated with elevated work environments and dense urban construction activities. High-altitude physiological monitoring components may account for atmospheric pressure variations and their effects on worker stress responses at significant building heights. Urban noise cancellation systems may be calibrated to filter traffic noise, emergency vehicle sirens, and adjacent construction activities typical of dense metropolitan environments. Multi-shift scheduling interfaces may accommodate extended construction hours common in urban projects while ensuring equitable therapeutic access across day, evening, and night work crews. The system may integrate with building management systems to coordinate therapeutic sessions with construction elevator schedules and material delivery operations.

For remote infrastructure development site deployments, the system 100 may be configured with enhanced self-sufficiency and communication capabilities to operate in locations with limited utilities and support infrastructure. Satellite communication interfaces may enable remote monitoring and technical support when cellular networks are unavailable or unreliable. Extended battery backup systems may provide prolonged operation during power outages common in remote locations. Weather-hardened enclosures may protect sensitive components from extreme environmental conditions including temperature variations, precipitation, and wind exposure typical of remote construction sites. The system may include enhanced air filtration to address dust and particulate matter from unpaved access roads and exposed soil conditions.

In residential development multi-phase deployment scenarios, the system 100 may be configured for sequential relocation between development phases while maintaining continuity of therapeutic services for construction crews. Phase transition protocols may enable rapid system relocation between completed and active construction areas without service interruption. Community integration features may minimize noise and visual impact on adjacent residential areas while maintaining full therapeutic functionality. Scalable deployment configurations may accommodate varying crew sizes across different development phases from initial site preparation through final landscaping activities. The system may coordinate with residential development scheduling to provide therapeutic support during high-stress construction activities such as foundation work and utility installation.

For renovation project deployments in operational facilities, the system 100 may be configured to provide therapeutic services while minimizing disruption to ongoing facility operations. Operational facility integration protocols may coordinate therapeutic sessions with existing facility schedules, security systems, and occupancy patterns. Contamination prevention systems may provide enhanced air filtration and sanitization to prevent construction-related contaminants from affecting facility operations. Noise isolation enhancements may ensure therapeutic sessions remain effective despite ongoing facility activities while preventing construction noise from disrupting facility operations. The system may include specialized access controls to distinguish between construction workers and facility occupants.

In industrial construction deployments with safety-critical stress monitoring, the system 100 may be configured with enhanced biometric monitoring and emergency response capabilities to address high-risk work environments. Critical stress threshold monitoring may implement more stringent physiological parameters and automated alerts for workers in safety-sensitive positions such as crane operators, welders, and electrical technicians. Enhanced emergency response integration may provide direct communication with site safety personnel and emergency medical services when critical stress levels are detected. Pre-shift fitness assessment protocols may evaluate worker stress levels before beginning safety-critical tasks and recommend therapeutic interventions when elevated stress could compromise safety performance. The system may maintain detailed stress monitoring logs for safety compliance documentation and incident investigation support.

### IV. Hardware Architecture

Embodiments of the present disclosure provide a hardware and software platform operative as a distributed system of modules and computing elements.

Platform 100 may be embodied as, for example, but not be limited to, a website, a web application, a desktop application, a backend application, and a mobile application compatible with a computing device 400. The computing device 400 may comprise, but not be limited to, the following:
Mobile computing device, such as, but is not limited to, a laptop, a tablet, a smartphone, a drone, a wearable, an embedded device, a handheld device, an Arduino, an industrial device, or a remotely operable recording device;
A supercomputer, an exascale supercomputer, a mainframe, or a quantum computer;
A minicomputer, wherein the minicomputer computing device comprises, but is not limited to, an IBM AS400/iSeries/System I, A DEC VAX/PDP, an HP3000, a Honeywell-Bull DPS, a Texas Instruments TI-990, or a Wang Laboratories VS Series;
A microcomputer, wherein the microcomputer computing device comprises, but is not limited to, a server, wherein a server may be rack-mounted, a workstation, an industrial device, a raspberry pi, a desktop, or an embedded device;

Platform 100 may be hosted on a centralized server or a cloud computing service. Although method 300 has been described to be performed by a computing device 400, it should be understood that, in some embodiments, different operations may be performed by a plurality of the computing devices 400 in operative communication on at least one network.

Embodiments of the present disclosure may comprise a system having a central processing unit (CPU) 420, a bus 430, a memory unit 440, a power supply unit (PSU) 450, and one or more Input/Output (I/O) units. The CPU 420 coupled to the memory unit 440 and the plurality of I/O units 460 via the bus 430, all of which are powered by the PSU 450. It should be understood that, in some embodiments, each disclosed unit may actually be a plurality of such units for redundancy, high availability, and/or performance purposes. The combination of the presently disclosed units is configured to perform the stages of any method disclosed herein.

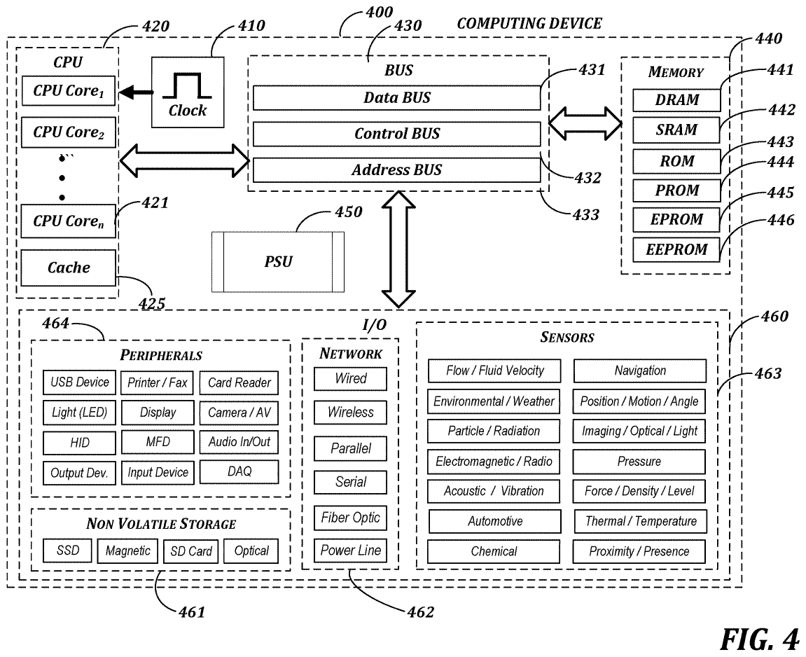

FIG. 4 is a block diagram of a system including computing device 400. Consistent with an embodiment of the disclosure, the aforementioned CPU 420, the bus 430, the memory unit 440, a PSU 450, and the plurality of I/O units 460 may be implemented in a computing device, such as computing device 400 of FIG. 4. Any suitable combination of hardware, software, or firmware may be used to implement the aforementioned units. For example, the CPU 420, the bus 430, and the memory unit 440 may be implemented with computing device 400 or any of other computing devices 400, in combination with computing device 400. The aforementioned system, device, and components are examples and other systems, devices, and components may comprise the aforementioned CPU 420, the bus 430, and the memory unit 440, consistent with embodiments of the disclosure.

At least one computing device 400 may be embodied as any of the computing elements illustrated in all of the attached figures. A computing device 400 does not need to be electronic, nor even have a CPU 420, nor bus 430, nor memory unit 440. The definition of the computing device 400 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 400, especially if the processing is purposeful.

With reference to FIG. 4, a system consistent with an embodiment of the disclosure may include a computing device, such as computing device 400. In some configurations, the computing device 400 may include at least one clock module 410, at least one CPU 420, at least one bus 430, and at least one memory unit 440, at least one PSU 450, and at least one I/O 460 module, wherein I/O module may be comprised of, but not limited to a non-volatile storage sub-module 461, a communication sub-module 462, a sensors sub-module 463, and a peripherals sub-module 464.

In a system consistent with an embodiment of the disclosure, the computing device 400 may include the clock module 410, known to a person having ordinary skill in the art as a clock generator, which produces clock signals. Clock signals may oscillate between a high state and a low state at a controllable rate, and may be used to synchronize or coordinate actions of digital circuits. Most integrated circuits (ICs) of sufficient complexity use a clock signal in order to synchronize different parts of the circuit, cycling at a rate slower than the worst-case internal propagation delays. One well-known example of the aforementioned integrated circuit is the CPU 420, the central component of modern computers, which relies on a clock signal. The clock 410 can comprise a plurality of embodiments, such as, but not limited to, a single-phase clock which transmits all clock signals on effectively 1 wire, a two-phase clock which distributes clock signals on two wires, each with non-overlapping pulses, and a four-phase clock which distributes clock signals on 4 wires.

Many computing devices 400 may use a “clock multiplier” which multiplies a lower frequency external clock to the appropriate clock rate of the CPU 420. This allows the CPU 420 to operate at a much higher frequency than the rest of the computing device 400, which affords performance gains in situations where the CPU 420 does not need to wait on an external factor (like memory 440 or input/output 460). Some embodiments of the clock 410 may include dynamic frequency change, where, the time between clock edges can vary widely from one edge to the next and back again.

In a system consistent with an embodiment of the disclosure, the computing device 400 may include the CPU 420 comprising at least one CPU Core 421. In other embodiments, the CPU 420 may include a plurality of identical CPU cores 421, such as, but not limited to, homogeneous multi-core systems. It is also possible for the plurality of CPU cores 421 to comprise different CPU cores 421, such as, but not limited to, heterogeneous multi-core systems, big. LITTLE systems and some AMD accelerated processing units (APU). The CPU 420 reads and executes program instructions which may be used across many application domains, for example, but not limited to, general purpose computing, embedded computing, network computing, digital signal processing (DSP), and graphics processing (GPU). The CPU 420 may run multiple instructions on separate CPU cores 421 simultaneously. The CPU 420 may be integrated into at least one of a single integrated circuit die, and multiple dies in a single chip package. The single integrated circuit die and/or the multiple dies in a single chip package may contain a plurality of other elements of the computing device 400, for example, but not limited to, the clock 410, the bus 430, the memory 440, and I/O 460.

The CPU 420 may contain cache 422 such as but not limited to a level 1 cache, a level 2 cache, a level 3 cache, or combinations thereof. The cache 422 may or may not be shared amongst a plurality of CPU cores 421. The cache 422 sharing may comprise at least one of message passing and inter-core communication methods used for the at least one CPU Core 421 to communicate with the cache 422. The inter-core communication methods may comprise, but not be limited to, bus, ring, two-dimensional mesh, and crossbar. The aforementioned CPU 420 may employ symmetric multiprocessing (SMP) design.

The one or more CPU cores 421 may comprise soft microprocessor cores on a single field programmable gate array (FPGA), such as semiconductor intellectual property cores (IP Core). The architectures of the one or more CPU cores 421 may be based on at least one of, but not limited to, Complex Instruction Set Computing (CISC), Zero Instruction Set Computing (ZISC), and Reduced Instruction Set Computing (RISC). At least one performance-enhancing method may be employed by one or more of the CPU cores 421, for example, but not limited to Instruction-level parallelism (ILP) such as, but not limited to, superscalar pipelining, and Thread-level parallelism (TLP).

Consistent with the embodiments of the present disclosure, the aforementioned computing device 400 may employ a communication system that transfers data between components inside the computing device 400, and/or the plurality of computing devices 400. The aforementioned communication system will be known to a person having ordinary skill in the art as a bus 430. The bus 430 may embody internal and/or external hardware and software components, for example, but not limited to a wire, an optical fiber, various communication protocols, and/or any physical arrangement that provides the same logical function as a parallel electrical bus. The bus 430 may comprise at least one of a parallel bus, wherein the parallel bus carries data words in parallel on multiple wires; and a serial bus, wherein the serial bus carries data in bit-wise serial form. The bus 430 may embody a plurality of topologies, for example, but not limited to, a multidrop/electrical parallel topology, a daisy chain topology, and connected by switched hubs, such as a USB bus. The bus 430 may comprise a plurality of embodiments, for example, but not limited to:
Internal Data Bus (data Bus) 431/Memory Bus
Control bus 432
Address bus 433
System Management Bus (SMBus)
Front-Side-Bus (FSB)
External Bus Interface (EBI)
Local bus
Expansion bus
Lightning bus
Controller Area Network (CAN bus)
Camera Link
ExpressCard
Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.
Small Computer System Interface (SCSI)/Serial Attached SCSI (SAS)
HyperTransport
InfiniBand
RapidIO
Mobile Industry Processor Interface (MIPI)
Coherent Processor Interface (CAPI)
Plug-n-play
1—Wire
Peripheral Component Interconnect (PCI), including embodiments such as but not limited to, Accelerated Graphics Port (AGP), Peripheral Component Interconnect eXtended (PCI-X), Peripheral Component Interconnect Express (PCI-e) (e.g., PCI Express Mini Card, PCI Express M.2 [Mini PCIe v2], PCI Express External Cabling [ePCIe], and PCI Express OCuLink [Optical Copper{Cu} Link]), Express Card, AdvancedTCA, AMC, Universal IO, Thunderbolt/Mini DisplayPort, Mobile PCIe (M-PCIe), U.2, and Non-Volatile Memory Express (NVMe)/Non-Volatile Memory Host Controller Interface Specification (NVMHCIS).
Industry Standard Architecture (ISA), including embodiments such as, but not limited to Extended ISA (EISA), PC/XT-bus/PC/AT-bus/PC/104 bus (e.g., PC/104-Plus, PCI/104-Express, PCI/104, and PCI-104), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1394 Interface/Firewire, Thunderbolt, and eXtensible Host Controller Interface (xHCI).

Consistent with the embodiments of the present disclosure, the aforementioned computing device 400 may employ hardware integrated circuits that store information for immediate use in the computing device 400, known to persons having ordinary skill in the art as primary storage or memory 440. The memory 440 operates at high speed, distinguishing it from the non-volatile storage sub-module 461, which may be referred to as secondary or tertiary storage, which provides relatively slower-access to information but offers higher storage capacity. The data contained in memory 440, may be transferred to secondary storage via techniques such as, but not limited to, virtual memory and swap. The memory 440 may be associated with addressable semiconductor memory, such as integrated circuits consisting of silicon-based transistors, that may be used as primary storage or for other purposes in the computing device 400. The memory 440 may comprise a plurality of embodiments, such as, but not limited to volatile memory, non-volatile memory, and semi-volatile memory. It should be understood by a person having ordinary skill in the art that the following are non-limiting examples of the aforementioned memory:
Volatile memory, which requires power to maintain stored information, for example, but not limited to, Dynamic Random-Access Memory (DRAM) 441, Static Random-Access Memory (SRAM) 442, CPU Cache memory 425, Advanced Random-Access Memory (A-RAM), and other types of primary storage such as Random-Access Memory (RAM).
Non-volatile memory, which can retain stored information even after power is removed, for example, but not limited to, Read-Only Memory (ROM) 443, Programmable ROM (PROM) 444, Erasable PROM (EPROM) 445, Electrically Erasable PROM (EEPROM) 446 (e.g., flash memory and Electrically Alterable PROM [EAPROM]), Mask ROM (MROM), One Time Programmable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.
Semi-volatile memory may have limited non-volatile duration after power is removed but may lose data after said duration has passed. Semi-volatile memory provides high performance, durability, and other valuable characteristics typically associated with volatile memory, while providing some benefits of true non-volatile memory. The semi-volatile memory may comprise volatile and non-volatile memory, and/or volatile memory with a battery to provide power after power is removed. The semi-volatile memory may comprise, but is not limited to, spin-transfer torque RAM (STT-RAM).

Consistent with the embodiments of the present disclosure, the aforementioned computing device 400 may employ a communication system between an information processing system, such as the computing device 400, and the outside world, for example, but not limited to, human, environment, and another computing device 400. The aforementioned communication system may be known to a person having ordinary skill in the art as an Input/Output (I/O) module 460. The I/O module 460 regulates a plurality of inputs and outputs with regard to the computing device 400, wherein the inputs are a plurality of signals and data received by the computing device 400, and the outputs are the plurality of signals and data sent from the computing device 400. The I/O module 460 interfaces with a plurality of hardware, such as, but not limited to, non-volatile storage 461, communication devices 462, sensors 463, and peripherals 464. The plurality of hardware is used by at least one of, but not limited to, humans, the environment, and another computing device 400 to communicate with the present computing device 400. The I/O module 460 may comprise a plurality of forms, for example, but not limited to channel I/O, port mapped I/O, asynchronous I/O, and Direct Memory Access (DMA).

Consistent with the embodiments of the present disclosure, the aforementioned computing device 400 may employ a non-volatile storage sub-module 461, which may be referred to by a person having ordinary skill in the art as one of secondary storage, external memory, tertiary storage, off-line storage, and auxiliary storage. The non-volatile storage sub-module 461 may not be accessed directly by the CPU 420 without using an intermediate area in the memory 440. The non-volatile storage sub-module 461 may not lose data when power is removed and may be orders of magnitude less costly than storage used in memory 440. Further, the non-volatile storage sub-module 461 may have a slower speed and higher latency than in other areas of the computing device 400. The non-volatile storage sub-module 461 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (461) may comprise a plurality of embodiments, such as, but not limited to:
Optical storage, for example, but not limited to, Compact Disk (CD) (CD-ROM/CD-R/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory
Holographic data storage such as Holographic Versatile Disk (HVD).
Molecular Memory
Deoxyribonucleic Acid (DNA) digital data storage

Consistent with the embodiments of the present disclosure, the computing device 400 may employ a communication sub-module 462 as a subset of the I/O module 460, which may be referred to by a person having ordinary skill in the art as at least one of, but not limited to, a computer network, a data network, and a network. The network may allow computing devices 400 to exchange data using connections, which may also be known to a person having ordinary skill in the art as data links, which may include data links between network nodes. The nodes may comprise networked computer devices 400 that may be configured to originate, route, and/or terminate data. The nodes may be identified by network addresses and may include a plurality of hosts consistent with the embodiments of a computing device 400. Examples of computing devices that may include a communication sub-module 462 include, but are not limited to, personal computers, phones, servers, drones, and networking devices such as, but not limited to, hubs, switches, routers, modems, and firewalls.

Two nodes can be considered networked together when one computing device 400 can exchange information with the other computing device 400, regardless of any direct connection between the two computing devices 400. The communication sub-module 462 supports a plurality of applications and services, such as, but not limited to World Wide Web (WWW), digital video and audio, shared use of application and storage computing devices 400, printers/scanners/fax machines, email/online chat/instant messaging, remote control, distributed computing, etc. The network may comprise one or more transmission mediums, such as, but not limited to conductive wire, fiber optics, and wireless signals. The network may comprise one or more communications protocols to organize network traffic, wherein application-specific communications protocols may be layered, and may be known to a person having ordinary skill in the art as being improved for carrying a specific type of payload, when compared with other more general communications protocols. The plurality of communications protocols may comprise, but are not limited to, IEEE 802, ethernet, Wireless LAN (WLAN/Wi-Fi), Internet Protocol (IP) suite (e.g., TCP/IP, UDP, Internet Protocol version 4 [IPv4], and Internet Protocol version 6 [IPv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], Integrated Digital Enhanced Network [IDEN], Long Term Evolution [LTE], LTE-Advanced [LTE-A], and fifth generation [5G] communication protocols).

The communication sub-module 462 may comprise a plurality of size, topology, traffic control mechanisms and organizational intent policies. The communication sub-module 462 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Wherein cellular systems embody technologies such as, but not limited to, 3G,4G (such as WiMAX and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line Communications

The aforementioned network may comprise a plurality of layouts, such as, but not limited to, bus networks such as Ethernet, star networks such as Wi-Fi, ring networks, mesh networks, fully connected networks, and tree networks. The network can be characterized by its physical capacity or its organizational purpose. Use of the network, including user authorization and access rights, may differ according to the layout of the network. The characterization may include, but is not limited to a nanoscale network, a Personal Area Network (PAN), a Local Area Network (LAN), a Home Area Network (HAN), a Storage Area Network (SAN), a Campus Area Network (CAN), a backbone network, a Metropolitan Area Network (MAN), a Wide Area Network (WAN), an enterprise private network, a Virtual Private Network (VPN), and a Global Area Network (GAN).

Consistent with the embodiments of the present disclosure, the aforementioned computing device 400 may employ a sensors sub-module 463 as a subset of the I/O 460. The sensors sub-module 463 comprises at least one of the device, module, or subsystem whose purpose is to detect events or changes in its environment and send the information to the computing device 400. Sensors may be sensitive to the property they are configured to measure, may not be sensitive to any property not measured but be encountered in its application, and may not significantly influence the measured property. The sensors sub-module 463 may comprise a plurality of digital devices and analog devices, wherein if an analog device is used, an Analog to Digital (A-to-D) converter must be employed to interface the said device with the computing device 400. The sensors may be subject to a plurality of deviations that limit sensor accuracy. The sensors sub-module 463 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:
Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nanosensors).
Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.
Acoustic, sound and vibration sensors, such as, but not limited to, microphone, lace sensors such as a guitar pickup, seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, moisture alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermoluminescent dosimeter.
Navigation sensors, such as, but not limited to, airspeed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as but not limited to, accelerometer, displacement sensor, flex sensor, free-fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, colorimeter, contact image sensor, electro-optical sensor, infra-red sensor, kinetic inductance detector, LED configured as a light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photoswitch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezocapacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 400 may employ a peripherals sub-module 464 as a subset of the I/O 460. The peripheral sub-module 464 comprises ancillary devices used to put information into and get information out of the computing device 400. There are 3 categories of devices comprising the peripheral sub-module 464, which exist based on their relationship with the computing device 400, input devices, output devices, and input/output devices. Input devices send at least one of data and instructions to the computing device 400. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to the position of a mouse.
The number of degrees of freedom involved, such as, but not limited to, two-dimensional mice and three-dimensional mice used for Computer-Aided Design (CAD) applications.

Output devices provide output from the computing device 400. Output devices convert electronically generated information into a form that can be presented to humans. Input/output devices that perform both input and output functions. It should be understood by a person having ordinary skill in the art that the ensuing are non-limiting embodiments of the aforementioned peripheral sub-module 464:
Input Devices
   Human Interface Devices (hid), Such As, but Not Limited to, Pointing Device (e.g., mouse, touchpad, joystick, touchscreen, game controller/gamepad, remote, light pen, light gun, infrared remote, jog dial, shuttle, and knob), keyboard, graphics tablet, digital pen, gesture recognition devices, magnetic ink character recognition, Sip-and-Puff (SNP) device, and Language Acquisition Device (LAD).
   High degree of freedom devices, that require up to six degrees of freedom such as, but not limited to, camera gimbals, Cave Automatic Virtual Environment (CAVE), and virtual reality systems.
   Video Input devices are used to digitize images or video from the outside world into the computing device 400. The information can be stored in a multitude of formats depending on the user's requirement. Examples of types of video input devices include, but are not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.
   Audio input devices are used to capture sound. In some cases, an audio output device can be used as an input device to capture produced sound. Audio input devices allow a user to send audio signals to the computing device 400 for at least one of processing, recording, and carrying out commands. Devices such as microphones allow users to speak to the computer to record a voice message or navigate software. Aside from recording, audio input devices are also used with speech recognition software. Examples of types of audio input devices include, but not limited to microphone, Musical Instrumental Digital Interface (MIDI) devices such as, but not limited to a keyboard, and headset.
   Data AcQuisition (DAQ) devices convert at least one of analog signals and physical parameters to digital values for processing by the computing device 400. Examples of DAQ devices may include, but not limited to, Analog to Digital Converter (ADC), data logger, signal conditioning circuitry, multiplexer, and Time to Digital Converter (TDC).
Output Devices may further comprise, but not be limited to:
   Display devices may convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).
   Printers, such as, but not limited to, inkjet printers, laser printers, 3D printers, solid ink printers, and plotters.
   Audio and Video (AV) devices, such as, but not limited to, speakers, headphones, amplifiers, and lights, which include lamps, strobes, DJ lighting, stage lighting, architectural lighting, special effect lighting, and lasers.
   Other devices such as Digital to Analog Converter (DAC)
Input/Output Devices may further comprise, but not be limited to, touchscreens, networking devices (e.g., devices disclosed in network sub-module 462), data storage devices (non-volatile storage 461), facsimile (FAX), and graphics/sound cards.

All rights, including copyrights in the code included herein, are vested in and the property of the Applicant. The Applicant retains and reserves all rights in the code included herein, and grants permission to reproduce the material only in connection with the reproduction of the granted patent and for no other purpose.

### V. Aspects

The following discloses various Aspects of the present disclosure. The various Aspects are not to be construed as patent claims unless the language of the Aspect appears as a patent claim. The Aspects describe various non-limiting embodiments of the present disclosure.

Aspect 1. An apparatus for providing on-site mental health therapy for construction workers, the apparatus comprising: a portable sound therapy booth comprising a modular frame, glass panels, and antimicrobial interior surfaces; a sound system disposed within the booth and configured to deliver therapeutic audio content; a visual projection system disposed within the booth and configured to display synchronized relaxation imagery; a plurality of contactless biometric sensors configured to monitor vital signs of a user without physical contact; and a controller communicatively coupled to the sound system, the visual projection system, and the plurality of contactless biometric sensors, wherein the controller is configured to adjust the therapeutic audio content and the synchronized relaxation imagery based on biometric data received from the plurality of contactless biometric sensors.

Aspect 2. The apparatus of Aspect 1, further comprising: an automated sanitization system comprising ultraviolet light components and sanitizing mist dispensers, wherein the automated sanitization system is configured to activate upon detection of booth vacancy after a user session.

Aspect 3. The apparatus of Aspect 1, wherein the plurality of contactless biometric sensors comprise: a thermal imaging camera configured to detect body temperature variations; a heart rate sensor configured to monitor cardiovascular activity; and a respiration sensor configured to monitor breathing patterns.

Aspect 4. The apparatus of Aspect 1, further comprising: an environmental control system configured to regulate temperature, humidity, and ventilation within the booth; and noise-cancellation hardware integrated into the booth structure to minimize external construction site noise.

Aspect 5. The apparatus of Aspect 1, wherein the controller is further configured to: store anonymized biometric data; analyze the anonymized biometric data to identify stress patterns; generate personalized therapy recommendations based on the identified stress patterns; and transmit encrypted session summary reports to an external occupational health monitoring system.

Aspect 6. A system for providing mental health therapy in high-stress work environments, the system comprising: a portable relaxation booth comprising a weatherproof exterior shell and antimicrobial interior components; a multi-sensory therapy delivery unit disposed within the booth, the multi-sensory therapy delivery unit comprising audio speakers and a visual display; a non-invasive biometric monitoring array comprising at least one contactless sensor configured to detect physiological indicators of stress; a sanitization subsystem comprising automated cleaning components; and a control unit operatively connected to the multi-sensory therapy delivery unit, the non-invasive biometric monitoring array, and the sanitization subsystem, wherein the control unit is configured to: receive real-time biometric data from the non-invasive biometric monitoring array; select therapeutic content based on the real-time biometric data; and activate the sanitization subsystem between user sessions.

Aspect 7. The system of Aspect 6, wherein the portable relaxation booth further comprises: an ergonomic seating element; soft lighting components; and noise-isolation materials integrated into the booth structure.

Aspect 8. The system of Aspect 6, wherein the non-invasive biometric monitoring array comprises: a thermal imaging sensor configured to detect facial temperature patterns; an optical heart rate monitor configured to detect pulse rate through skin color variations; and a motion sensor configured to detect breathing patterns through chest movement.

Aspect 9. The system of Aspect 6, wherein the control unit further comprises: a secure data storage module configured to store anonymized user session data; a machine learning processor configured to analyze the anonymized user session data to identify effectiveness patterns of different therapeutic content; and a wireless communication module configured to transmit encrypted aggregated wellness metrics to an external occupational health management system.

Aspect 10. The system of Aspect 6, further comprising: a user authentication interface configured to control access to the portable relaxation booth; a session management module configured to enforce predetermined time limits for therapy sessions; and an emergency alert system configured to detect abnormal biometric readings indicating potential health emergencies.

Aspect 11. A mental health therapy system for construction sites, the system comprising: a sound therapy booth comprising a modular structure adapted for deployment in construction environments; a biometric sensing subsystem comprising a plurality of non-contact sensors positioned within the sound therapy booth, wherein the non-contact sensors are configured to collect physiological data from a user without physical contact; a multi-modal therapy subsystem comprising audio components and visual display components integrated within the sound therapy booth; an automated cleaning subsystem comprising sanitization elements configured to disinfect interior surfaces of the sound therapy booth; and a control subsystem communicatively coupled to the biometric sensing subsystem, the multi-modal therapy subsystem, and the automated cleaning subsystem, wherein the control subsystem is configured to: process the physiological data to determine stress indicators; select and deliver customized therapeutic content through the multi-modal therapy subsystem based on the determined stress indicators; and initiate a cleaning cycle using the automated cleaning subsystem when the sound therapy booth is unoccupied.

Aspect 12. The mental health therapy system of Aspect 11, wherein the modular structure comprises: a weatherproof exterior shell designed to withstand construction site conditions; noise-dampening materials integrated into walls of the sound therapy booth; and antimicrobial interior surface materials that resist contamination in dusty environments.

Aspect 13. The mental health therapy system of Aspect 11, wherein the biometric sensing subsystem comprises: a thermal imaging sensor configured to detect facial temperature patterns indicative of stress; a camera-based heart rate sensor that analyzes subtle skin color variations to determine pulse rate; and a respiration monitoring sensor that detects breathing patterns through non-contact measurement of chest movements.

Aspect 14. The mental health therapy system of Aspect 11, wherein the control subsystem further comprises: a secure data storage module that stores anonymized biometric data; a machine learning processor that analyzes historical session data to improve therapeutic content selection; and a secure communication module that transmits encrypted workforce wellness metrics to external occupational health systems while maintaining individual privacy.

Aspect 15. The mental health therapy system of Aspect 11, further comprising an environmental regulation subsystem that controls temperature, humidity, and air quality within the sound therapy booth, wherein the environmental regulation subsystem adjusts environmental parameters based on external construction site conditions and user preferences.

Aspect 16. A method for providing on-site mental health therapy for construction workers, the method comprising: detecting presence of a user in a portable sound therapy booth; initiating a therapy session upon detecting the user presence; collecting biometric data from the user using a plurality of contactless biometric sensors; analyzing the biometric data to determine stress indicators of the user; selecting therapeutic audio content and synchronized relaxation imagery based on the determined stress indicators; delivering the therapeutic audio content through a sound system disposed within the portable sound therapy booth; displaying the synchronized relaxation imagery through a visual projection system disposed within the portable sound therapy booth; monitoring changes in the biometric data during the therapy session; adjusting the therapeutic audio content and the synchronized relaxation imagery in real-time based on the monitored changes in the biometric data; and activating an automated sanitization system upon detecting vacancy of the portable sound therapy booth after completion of the therapy session.

Aspect 17. The method of Aspect 16, further comprising: creating a user profile for the user; storing anonymized biometric data and therapy session information associated with the user profile; analyzing historical therapy session data to identify patterns in stress reduction effectiveness; and optimizing subsequent therapy sessions based on the identified patterns.

Aspect 18. The method of Aspect 16, wherein collecting biometric data comprises: capturing thermal images of the user to detect body temperature variations; monitoring cardiovascular activity through a contactless heart rate sensor; and tracking breathing patterns through a respiration sensor without physical contact with the user.

Aspect 19. The method of Aspect 16, further comprising: regulating environmental conditions within the portable sound therapy booth by adjusting temperature, humidity, and ventilation parameters; activating noise-cancellation hardware to minimize external construction site noise; and adjusting soft lighting components to create a calming environment within the portable sound therapy booth.

Aspect 20. The method of Aspect 16, further comprising: generating an encrypted session summary report containing anonymized biometric data and therapy effectiveness metrics; transmitting the encrypted session summary report to an external occupational health monitoring system; and generating aggregate wellness metrics across multiple users while maintaining individual privacy.

Aspect 21. A portable mental health support apparatus for construction sites, the apparatus comprising: a booth structure comprising a durable frame, weatherproof exterior panels, and antimicrobial interior surfaces; a data collection system comprising contactless sensors configured to monitor user stress indicators without physical contact; a multi-sensory relaxation system comprising audio components delivering construction-themed sound therapy and visual components displaying synchronized calming imagery; a secure computing module configured to process collected biometric data while maintaining user privacy; and a usage monitoring system configured to enforce predetermined session time limits to prevent booth misuse.

Aspect 22. The portable mental health support apparatus of Aspect 21, wherein the data collection system comprises: a thermal imaging sensor configured to detect facial temperature variations; an optical sensor configured to measure heart rate variability through skin tone analysis; and a motion detection sensor configured to analyze breathing patterns.

Aspect 23. The portable mental health support apparatus of Aspect 21, wherein the multi-sensory relaxation system further comprises: construction-themed visual elements integrated into relaxation imagery; industry-specific audio therapy content designed for construction worker stress patterns; and ergonomic seating configured to accommodate users wearing work gear.

Aspect 24. The portable mental health support apparatus of Aspect 21, further comprising: a modular design allowing for transportation between different construction sites; anchoring components configured to secure the booth to various site surfaces; and protective elements configured to shield the apparatus from construction debris.

Aspect 25. The portable mental health support apparatus of Aspect 21, further comprising: a machine learning processor configured to analyze anonymized aggregate user data; a trending module configured to identify patterns in workforce stress levels; and a reporting system configured to generate site-specific mental health analytics for occupational health program assessment.

Aspect 26. A method for monitoring and improving mental health of construction workers, the method comprising: detecting a construction worker entering a portable relaxation booth positioned at a construction site; authenticating the construction worker through a user authentication interface; activating a non-invasive biometric monitoring system comprising contactless sensors within the portable relaxation booth; collecting real-time physiological data from the construction worker using the contactless sensors; analyzing the real-time physiological data to determine current stress levels of the construction worker; selecting therapeutic content from a content library based on the determined stress levels; delivering multi-sensory therapy to the construction worker through a combination of sound therapy via audio speakers and visual relaxation via a display system; continuously monitoring changes in the physiological data during the therapy session; dynamically adjusting the therapeutic content based on the monitored changes in the physiological data; storing anonymized session data in a secure data storage module; and initiating an automated sanitization sequence when the construction worker exits the portable relaxation booth.

Aspect 27. The method of Aspect 26, further comprising: establishing a baseline stress profile for the construction worker based on historical biometric data; comparing current physiological data to the baseline stress profile; identifying deviations from the baseline stress profile; and customizing the therapeutic content based on the identified deviations.

Aspect 28. The method of Aspect 26, wherein the contactless sensors comprise: a thermal imaging camera detecting facial temperature patterns; an optical sensor detecting heart rate through skin color variations; and a motion sensor detecting respiration rate through subtle chest movements.

Aspect 29. The method of Aspect 26, further comprising: generating an encrypted session report containing anonymized biometric data and therapy effectiveness metrics; transmitting the encrypted session report to an occupational health monitoring system; and aggregating data across multiple sessions to identify stress patterns among construction workers while maintaining individual privacy.

Aspect 30. A modular mental health therapy booth for construction environments, the booth comprising: a weatherproof exterior shell configured to withstand construction site conditions; a modular frame comprising interconnectable components allowing for transportation between construction sites; antimicrobial interior surfaces resistant to contamination in dusty environments; an audio system delivering therapeutic sound content calibrated for stress reduction; a visual display system projecting synchronized relaxation imagery; a biometric sensing array comprising contactless sensors configured to monitor physiological indicators of stress; an automated sanitization system comprising ultraviolet light components and antimicrobial mist dispensers; a secure computing module processing biometric data while maintaining user privacy; and a control system communicatively coupled to the audio system, the visual display system, the biometric sensing array, and the automated sanitization system.

Aspect 31. The modular mental health therapy booth of Aspect 30, further comprising: noise-cancellation hardware integrated into the booth structure to minimize external construction site noise; an environmental control system regulating temperature, humidity, and ventilation within the booth; and an ergonomic seating element designed to accommodate construction workers wearing safety gear.

Aspect 32. The modular mental health therapy booth of Aspect 30, wherein the biometric sensing array comprises: a thermal imaging camera configured to detect facial temperature patterns indicative of stress; a contactless heart rate monitor utilizing light reflection analysis; and a respiratory pattern detector utilizing motion sensing technology.

Aspect 33. The modular mental health therapy booth of Aspect 30, further comprising: a data analytics system configured to: collect anonymized biometric data from multiple users; identify stress trends across construction site personnel; evaluate effectiveness of different therapeutic content; and generate reports for occupational health program assessment.

Aspect 34. The modular mental health therapy booth of Aspect 30, further comprising: a secure wireless communication module transmitting encrypted data to external systems; a user authentication system controlling booth access; and a session management system enforcing predetermined time limits for therapy sessions.

Aspect 35. A system for data-driven mental health support in construction environments, the system comprising: a portable therapy booth positioned at a construction site; a multi-sensory therapy delivery subsystem within the booth comprising audio components and visual display components; a contactless biometric monitoring subsystem comprising sensors configured to collect physiological data without physical contact; a data processing subsystem configured to: analyze collected physiological data to identify stress indicators; select therapeutic content based on identified stress indicators; track changes in physiological responses during therapy sessions; store anonymized session data; and generate mental health analytics for workforce wellness assessment; a sanitization subsystem configured to automatically disinfect the booth between user sessions; and a user interface subsystem configured to facilitate booth access and therapy session management.

Aspect 36. The system of Aspect 35, wherein the portable therapy booth comprises: a modular structure allowing for relocation between construction sites; weatherproof exterior materials designed to withstand outdoor conditions; noise-isolation components reducing external construction noise; and antimicrobial interior surfaces.

Aspect 37. The system of Aspect 35, wherein the contactless biometric monitoring subsystem comprises: thermal imaging sensors detecting facial temperature patterns; optical sensors detecting heart rate variability through skin color analysis; and motion detection sensors analyzing breathing patterns.

Aspect 38. The system of Aspect 35, wherein the data processing subsystem further comprises: a machine learning processor analyzing historical session data to improve therapeutic content selection; a trend identification module detecting patterns in workforce stress levels; and a reporting module generating site-specific mental health analytics.

Aspect 39. The system of Aspect 35, further comprising: a secure communication module transmitting encrypted wellness metrics to external occupational health systems; an emergency alert system detecting abnormal biometric readings; and a power management system utilizing energy-efficient components compatible with construction site power infrastructure.

Aspect 40. A method for providing data-driven mental health therapy in construction environments, the method comprising: deploying a portable sound therapy booth at a construction site; establishing user profiles for construction workers accessing the booth; collecting baseline biometric data for each user through contactless sensors; delivering personalized sound therapy and visual relaxation content based on individual stress patterns; monitoring real-time physiological responses during therapy sessions; adapting therapeutic content based on detected physiological changes; sanitizing the booth automatically between user sessions; storing anonymized session data securely; analyzing aggregate data to identify stress trends across the workforce; generating reports on mental health patterns and therapy effectiveness; and refining therapeutic approaches based on analyzed data.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

1. An apparatus comprising:
a portable booth comprising a weatherproof exterior shell, a modular frame, and antimicrobial interior surfaces;
a sound system disposed within the portable booth and configured to deliver therapeutic audio content;
a visual display system disposed within the portable booth and configured to display relaxation imagery synchronized with the therapeutic audio content;
a haptic transducer array disposed within the portable booth and configured to deliver vibrotactile stimulation to a user within the portable booth;
an ambient lighting system disposed within the portable booth and configured to deliver chromatic and intensity-variable light output synchronized with the therapeutic audio content and the relaxation imagery;
a contactless biometric sensing array positioned within the portable booth and configured to collect physiological data from a user without physical contact with the user;
an automated sanitization system configured to activate upon detection of booth vacancy following a user session, the automated sanitization system comprising at least one of an ultraviolet light emitter and a sanitizing mist dispenser; and
a controller communicatively coupled to the sound system, the visual display system, the haptic transducer array, the ambient lighting system, the contactless biometric sensing array, and the automated sanitization system, the controller configured to:
receive the physiological data from the contactless biometric sensing array;
determine one or more stress indicators from the physiological data;
select therapeutic content based on the determined stress indicators;
deliver the selected therapeutic content through the sound system, the visual display system, the haptic transducer array, and the ambient lighting system in a coordinated, synchronized manner; and
dynamically adjust the therapeutic content in real time based on changes in the physiological data monitored during a user session.

2. The apparatus of claim 1, wherein the contactless biometric sensing array comprises:
a thermal imaging sensor configured to detect facial temperature patterns associated with physiological stress;
an optical sensor configured to measure heart rate by detecting skin color variations associated with blood volume changes; and
a motion sensor configured to detect respiration rate through non-contact measurement of chest movement.

3. The apparatus of claim 1, wherein the modular frame comprises interconnectable components configured to permit disassembly, transport, and reassembly of the portable booth at different deployment locations.

4. The apparatus of claim 1, wherein the automated sanitization system further comprises an air purification unit comprising a HEPA filter configured to remove airborne particulates from the booth interior during a sanitization cycle.

5. The apparatus of claim 1, wherein the controller is further configured to:
detect an abnormal biometric reading from the contactless biometric sensing array; and
generate an emergency alert in response to the detected abnormal biometric reading.

6. The apparatus of claim 1, further comprising a machine learning processor configured to:
analyze anonymized aggregate session data from a plurality of user sessions; and
update therapeutic content selection parameters based on the analyzed aggregate session data to improve stress reduction outcomes across subsequent sessions.

7. The apparatus of claim 1, wherein the sound system is further configured to deliver binaural beat audio content at frequencies selected based on the determined stress indicators.

8. The apparatus of claim 1, further comprising:
a session management module configured to enforce a predetermined session time limit; and
a user authentication interface configured to control access to the portable booth using a non-personally-identifying credential.

9. A method comprising:
detecting presence of a user within a portable booth deployed at a worksite;
collecting physiological data from the user using a plurality of contactless biometric sensors positioned within the portable booth, wherein the physiological data is collected without physical contact with the user;
determining one or more stress indicators from the collected physiological data;
selecting therapeutic audio content and synchronized visual content based on the determined stress indicators;
delivering the therapeutic audio content through a sound system disposed within the portable booth, displaying the synchronized visual content through a visual display system disposed within the portable booth, activating a haptic transducer array to deliver vibrotactile stimulation synchronized with the therapeutic audio content, and activating an ambient lighting system to deliver chromatic light output synchronized with the therapeutic audio content and the synchronized visual content;
monitoring changes in the physiological data during a therapy session;
adjusting at least one of the therapeutic audio content, the synchronized visual content, the vibrotactile stimulation, and the chromatic light output in real time based on the monitored changes; and
activating an automated sanitization system within the portable booth upon detecting vacancy of the portable booth following completion of the therapy session.

10. The method of claim 9, wherein collecting physiological data comprises:
capturing thermal images of the user to detect facial temperature patterns;
measuring heart rate by optically detecting skin color variations; and
detecting respiration rate through non-contact sensing of chest movement.

11. The method of claim 9, further comprising:
storing anonymized biometric data and session information in encrypted local storage;
generating an encrypted session summary report comprising the anonymized biometric data and therapy effectiveness metrics; and
transmitting the encrypted session summary report to an external occupational health monitoring system.

12. The method of claim 9, further comprising:
regulating temperature, humidity, and ventilation within the portable booth; and
activating active noise-cancellation hardware to attenuate external ambient noise within the portable booth interior.

13. The method of claim 9, further comprising:
analyzing anonymized aggregate physiological data from a plurality of user sessions using a machine learning processor; and
generating workforce wellness metrics identifying stress trends across a plurality of users while maintaining individual user privacy.

14. The method of claim 9, wherein activating the automated sanitization system comprises:
confirming vacancy of the portable booth using at least two independent occupancy detection modalities; and
sequentially activating an ultraviolet light emitter and a sanitizing mist dispenser to disinfect interior surfaces of the portable booth.

15. The method of claim 9, further comprising:
establishing a baseline stress profile for the user based on physiological data collected at the start of the therapy session; and
comparing physiological data collected during the therapy session to the baseline stress profile to quantify a measurable reduction in stress indicators achieved during the session.

16. The method of claim 9, wherein the therapy session comprises a predetermined duration of between two minutes and fifteen minutes, and wherein the portable booth is configured to attenuate external ambient noise to a level sufficient to permit delivery of the therapeutic audio content at a therapeutic efficacy level within a high-ambient-noise deployment environment.

17. A system comprising:
a portable booth comprising a weatherproof exterior shell, a modular frame configured for transport and reassembly between worksites, and antimicrobial interior surfaces;
a multi-sensory therapy delivery subsystem disposed within the portable booth and comprising a sound system, a visual display system, a haptic transducer array, and an ambient lighting system, the multi-sensory therapy delivery subsystem configured to deliver coordinated, synchronized therapeutic audio content, visual content, vibrotactile stimulation, and chromatic light output;
a contactless biometric monitoring subsystem comprising a plurality of non-contact sensors configured to collect physiological data from a user within the portable booth without physical contact with the user;
an automated sanitization subsystem configured to disinfect interior surfaces of the portable booth between user sessions;
a data processing subsystem configured to:
determine stress indicators from the physiological data;
select and deliver therapeutic content through the multi-sensory therapy delivery subsystem based on the determined stress indicators;
dynamically adjust the therapeutic content based on changes in the physiological data monitored during a therapy session;
store anonymized session data; and
generate workforce wellness metrics from the anonymized session data; and
a secure communication module configured to transmit encrypted workforce wellness metrics to an external occupational health system.

18. The system of claim 17, wherein the contactless biometric monitoring subsystem comprises a thermal imaging sensor, an optical heart rate sensor, and a motion-based respiration sensor, each configured to operate without physical contact with the user and without requiring removal of personal protective equipment by the user.

19. The system of claim 17, wherein the data processing subsystem further comprises a machine learning processor configured to analyze anonymized aggregate session data from a plurality of users and update therapeutic content selection parameters to improve measurable stress reduction outcomes across subsequent therapy sessions.

20. The system of claim 17, wherein the haptic transducer array comprises a left-side transducer and a right-side transducer, and wherein the data processing subsystem is further configured to activate the left-side transducer and the right-side transducer in an alternating bilateral stimulation pattern, the alternating bilateral stimulation pattern being synchronized with the therapeutic audio content delivered through the sound system.
